(19) United States
(12) Patent Application Publication
(10) Pub. No.: US 2009/0197900 A1
(43) Pub. Date: Aug. 6, 2009

(54) METHODS OF TREATING HEART FAILURE AND RENAL DYSFUNCTION IN INDIVIDUALS WITH AN ADENOSINE A1 RECEPTOR ANTAGONIST

(76) Inventor: Howard Dittrich, San Diego, CA (US)

Correspondence Address: MERCK AND CO., INC, P O BOX 2000, RAHWAY, NJ, 07065-0907, US

(21) Appl. No.: 12/058,129

(22) Filed: Mar. 28, 2008

Related U.S. Application Data
(60) Provisional application No. 60/908,942, filed on Mar. 29, 2007.

(51) Int. Cl. A61K 31/522; A61P 13/12

(52) U.S. Cl. 514/263.34

(57) ABSTRACT

Provided herein are methods of improving, maintaining and restoring renal function, treating heart failure, treating subjects with acute fluid overload, and slowing or reversing an existing or developing renal impairment in subjects with BNP levels of at least 400 pg/mL and/or NT-proBNP levels of at least about 1500 pg/mL by administering a therapeutically effective amount of an AA₁RA to the subject.

### RELATED APPLICATIONS

The present application claims priority to U.S. Provisional Application Ser. No. 60/908,942, filed on Mar. 29, 2007, by Dittrich, and entitled “METHODS OF TREATING HEART FAILURE AND RENAL DYSFUNCTION IN INDIVIDUALS WITH AN ADENOSINE A1 RECEPTOR ANTAGONIST,” the entire disclosure of which is herein incorporated by reference in its entirety.

### BACKGROUND OF THE INVENTION

1. Field of the Invention

The present invention relates to methods of treating heart failure and renal dysfunction using an adenosine A₁ receptor antagonist.

2. Description of the Related Art

Adenosine is involved in the regulation of renal haemodynamics, tubular reabsorption of fluid and solutes, and in renin release in kidneys. In contrast to other vascular beds, adenosine induces vasoconstriction in the kidney, thereby coupling renal perfusion to the metabolic rate of the organ.

Adenosine exerts its biologic functions through binding to different G-Protein Coupled Receptors (“GPCRs”), e.g., A₁, A_{2A}, A_{2B}, A₃ and A₄. The adenosine A₁ receptor regulates renal fluid balance, as well as excitatory glutamatergic neurotransmission, which contributes to its anticonvulsant activity. Antagonists to A₁ receptors (AA₁RAs) cause diuresis and natriuresis without major changes in glomerular filtration rate (“GFR”) and decrease afferent arteriolar pressure. Xanthine-derived adenosine A₁ receptor antagonists, such as KW-3902, are effective diuretics, renal-protectants, and bronchodilators, also lower the seizure threshold of individuals.

The chemical name of the AA₁RA KW-3902 is 8-(3-noradamantyl)-1,3-dipropylxanthine, also known as 3,7-dihydro-1,3-dipropyl-8-(3-tricyclo[3.3.1.0^{3,7}]nonyl)-1H-purine-2,6-dione, and its structure is

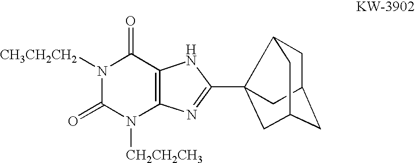

KW-3902 and related compounds have a diuretic effect, a renal-protecting effect, and a bronchodilatory effect. Further, KW-3902, when combined with a standard diuretic is beneficial to subjects who are refractory to standard therapy. KW-3902 also blocks the tubuloglomerular feedback (“TGF”) mechanism mediated by adenosine (via A₁ receptors) described above. This ultimately allows for increased GFR and improved renal function, which results in more fluid passing through the loop of Henle and the distal tubule. In addition, KW-3902 inhibits the reabsorption of sodium (and, therefore, water) in the proximal tubule, which results in diuresis. Furthermore, KW-3902 is an inhibitor of TGF, and can counteract the adverse effect of some diuretics, such as proximal diuretics, which activate or promote TGF. See, e.g., U.S. Pat. No. 5,290,782, and U.S. patent application Ser. Nos. 10/830,617 filed Apr. 23, 2004, Ser. No. 11/248,479 filed Oct. 11, 2005, Ser. No. 11/248,905 filed Oct. 11, 2005, and Ser. No. 11/464,665, filed Jun. 16, 2006 the entire disclosure of all of which are hereby incorporated by reference herein, including any drawings.

### SUMMARY OF THE INVENTION

Provided herein are methods of improving, maintaining and restoring renal function, in subjects in need thereof, methods of treating heart failure, methods of treating patients with acute fluid overload, and methods and slowing or reversing an existing or developing renal impairment (e.g., mild to severe renal impairment) in subjects determined to have BNP levels above about 400 pg/mL, e.g., above about 450 pg/ml, above about 460 pg/mL, above about 470 pg/mL, above about 480 pg/mL, above about 500 pg/mL or more, or any number in between and/or subjects determined to have NT-proBNP levels above about 1500 pg/ml, e.g., above about 1600 pg/mL, above about 1700 pg/mL, above about 1800 pg/mL, above about 1900 pg/mL, above about 2000 pg/mL or more, or any number in between.

In some embodiments, the subject can be provided a therapeutically effective amount of an AA₁RA, such as KW-3902 or a pharmaceutically acceptable salt, ester, amide, metabolite or prodrug thereof. Accordingly, the subject can be provided between 2.5 and 100 mg KW-3902 per day, preferably between 20 mg and 40 mg KW-3902 per day, and most preferably about 30 mg KW-3902 per day.

In some embodiments, the subject can have CHF and have impaired renal function. In some embodiments, the subjects with CHF can have a decreasing creatinine clearance rate, or an increase in serum creatinine levels. In some embodiments, the subject can have CHF and normal creatinine clearance and/or serum creatinine levels.

In some embodiments, the subject can be refractory to standard diuretic therapy, whereas in other embodiments, the subject is not refractory to standard diuretic therapy. In some embodiments, the subject can be provided a non-adenosine-modifying diuretic in addition to the AA₁RA, such as a proximal diuretic, a loop diuretic, or a distal diuretic. Preferably, the subject is provided furosemide.

### BRIEF DESCRIPTION OF THE DRAWINGS

FIG. 1 shows the percentage of subjects treated with 10 mg, 20 mg, 30 mg KW-3902 or placebo falling into the three different categories designated for the primary endpoint of the study described in Example 1: Success, Unchanged, and Failure.

FIG. 2 shows the percentages of a subgroup of subjects identified with BNP levels greater than 500 pmol, and/or NT-pro-BNP levels greater than 2000 pmol, treated with 10 mg, 20 mg, or 30 mg KW-3902, or placebo, falling into the three different categories designated for the primary endpoint of the study described in Example 1: Success, Unchanged, and Failure.

FIG. 3 shows the percentages of a subgroup of subjects identified with BNP levels greater than 500 pmol, and/or NT-pro-BNP levels greater than 2000 pmol, treated with 10 mg, 20 mg, or 30 mg KW-3902, as a combined total (“active”) or placebo (“placebo”) falling into the three different categories designated for the primary endpoint of the study described in Example 1: Success, Unchanged, and Failure.

FIG. 4 with acute CHF and mild to severe renal impairment requiring intravenous diuretic therapy shows the change in mean serum creatinine levels over the indicated time period in subjects following treatment with 10 mg, 20 mg, or 30 mg KW-3902, or placebo as described in Example 1.

FIG. 5 shows the change in mean serum creatinine levels over the indicated time period in a subgroup of subjects identified with BNP levels greater than 500 pmol, and/or NT-pro-BNP levels greater than 2000 pmol following treatment with 10 mg, 20 mg, or 30 mg KW-3902, or placebo as described in Example 1.

FIG. 6 shows the change in mean serum creatinine levels over the indicated time period in a subgroup subjects identified with BNP levels greater than 500 pmol, and/or NT-pro-BNP levels greater than 2000 pmol, following treatment with 10 mg, 20 mg, or 30 mg KW-3902, as a combined total (“active”) or placebo (“placebo”) as described in Example 1.

FIG. 7 shows the percentage of subjects with acute CHF and mild to severe renal impairment requiring intravenous diuretic therapy treated with 10 mg, 20 mg, or 30 mg KW-3902, or placebo that reported moderate or marked improvement in dyspnea, as described in Example 1.

FIG. 8 shows the percentages of a subgroup of subjects identified with BNP levels greater than 500 pmol, and/or NT-pro-BNP levels greater than 2000 pmol treated with 10 mg, 20 mg, or 30 mg KW-3902, or placebo that reported moderate or marked improvement in dyspnea, as described in Example 1.

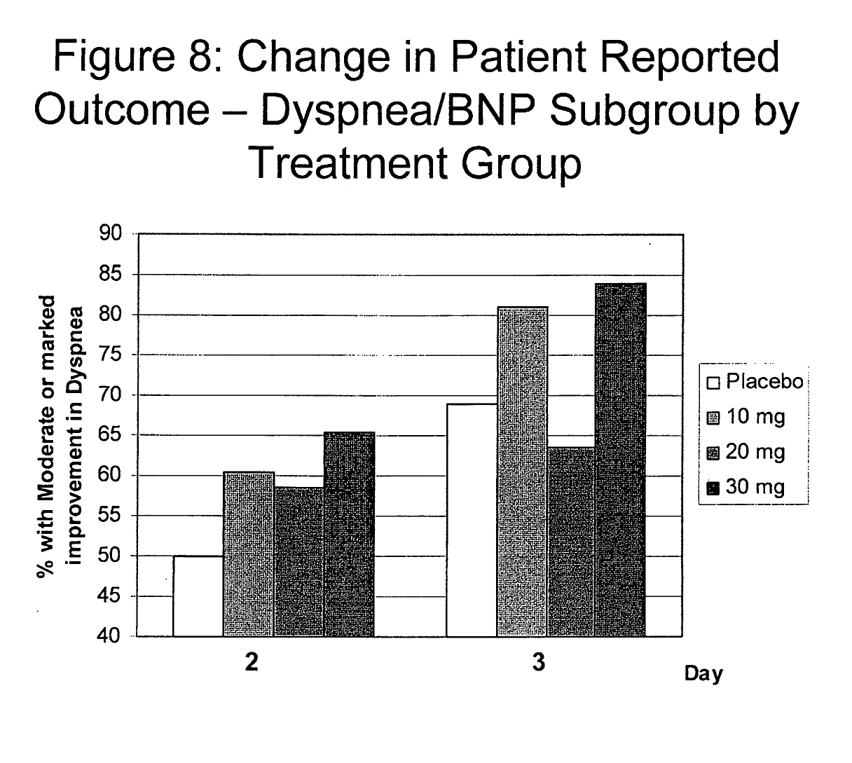

FIG. 9 shows the percentages of a subgroup of subjects identified with BNP levels greater than 500 pmol, and/or NT-pro-BNP levels greater than 2000 pmol treated with 10 mg, 20 mg, or 30 mg KW-3902, as a combined total (“active”) or placebo (“placebo”) that reported moderate or marked improvement in dyspnea, as described in Example 1.

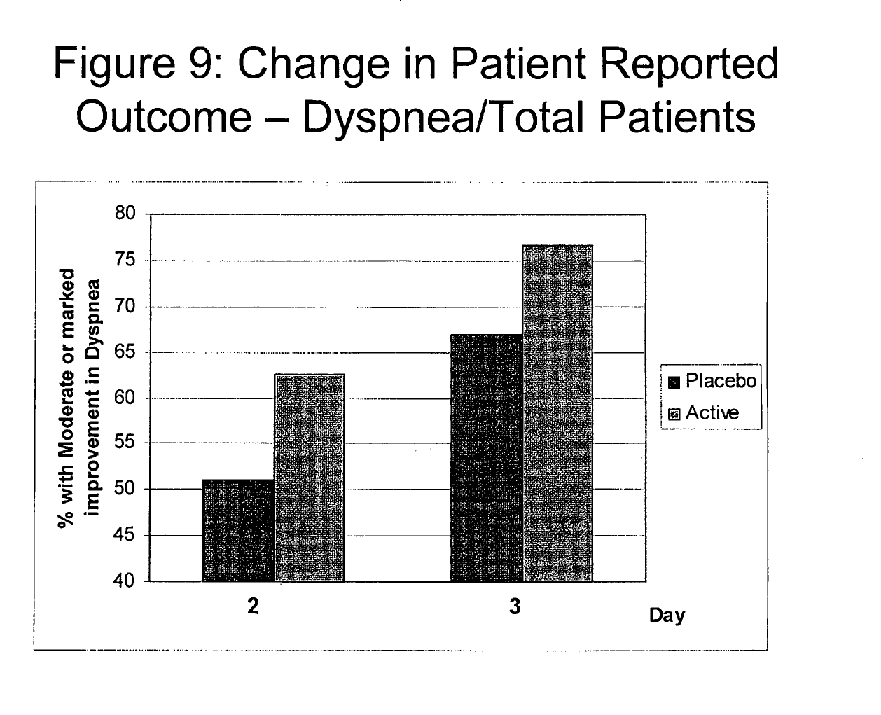

FIG. 10 shows the percentage of subjects with acute CHF and mild to severe renal impairment requiring intravenous diuretic therapy treated with 10 mg, 20 mg, or 30 mg KW-3902, or placebo falling into the “Success” category designated for the primary endpoint of the study over time, as described in Example 1.

FIG. 11 shows the percentage of a subgroup of subjects identified with BNP levels greater than 500 pmol, and/or NT-pro-BNP levels greater than 2000 pmol treated with 10 mg, 20 mg, or 30 mg KW-3902, as a combined total (“active”) or placebo (“placebo”) falling into the “Success” category designated for the primary endpoint of the study over time, as described in Example 1.

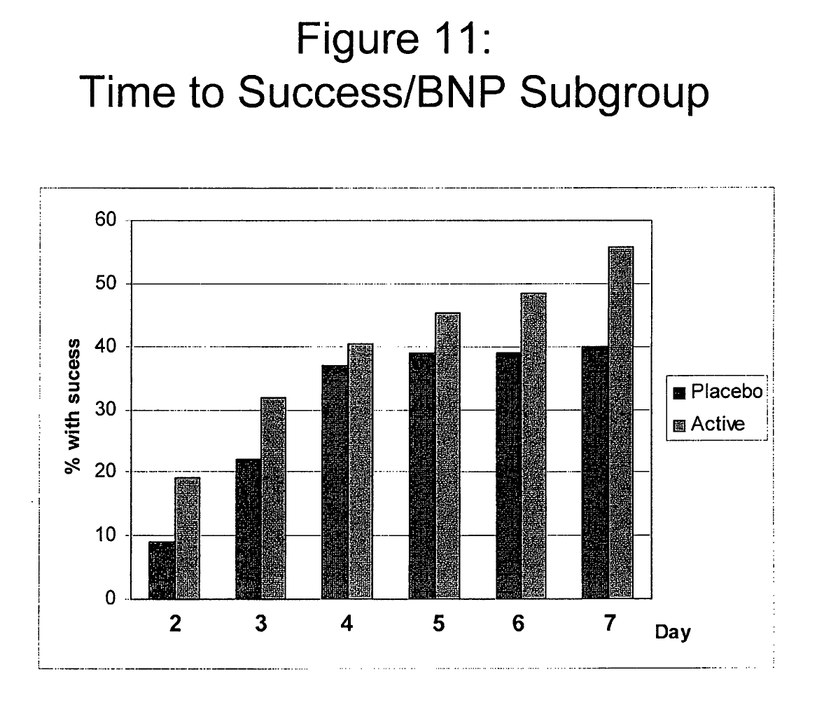

FIG. 12 shows the percentage of a subgroup of subjects with acute CHF treated with 10 mg, 20 mg, or 30 mg KW-3902 or placebo exhibiting worsening heart failure over the indicated time periods following treatment, as described in Example 1.

FIG. 13 shows the percentage of a subgroup of subjects identified with BNP levels greater than 500 pmol, and/or NT-pro-BNP levels greater than 2000 pmol treated with 10 mg, 20 mg, or 30 mg KW-3902, as a combined total (“active”) or placebo (“placebo”) exhibiting worsening heart failure over the indicated time periods following treatment, as described in Example 1.

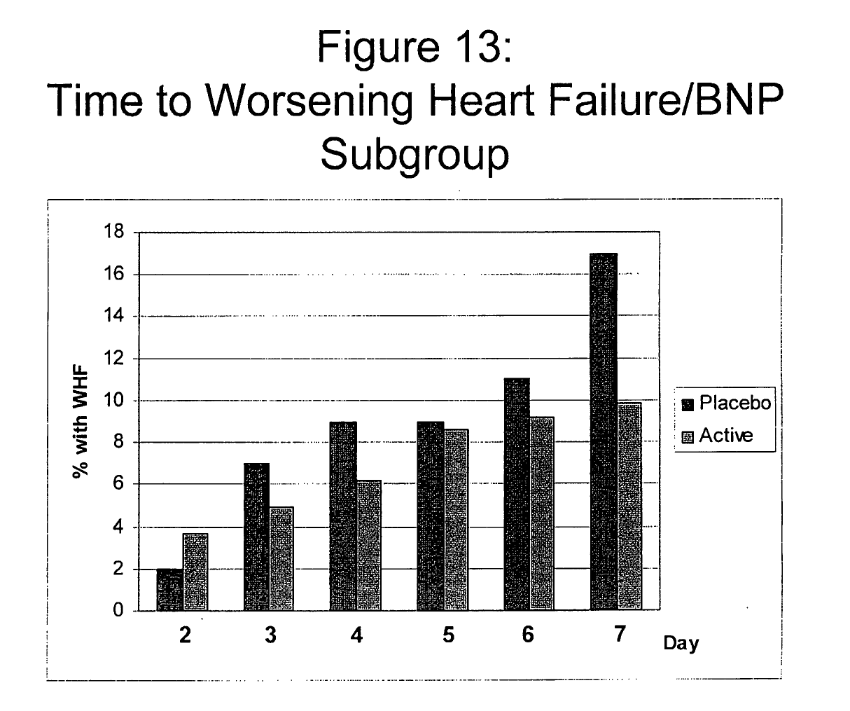

FIG. 14 shows the percentages of a subgroup of subjects identified with BNP levels greater than 500 pmol, and/or NT-pro-BNP levels greater than 2000 pmol, treated with 10 mg, 20 mg, or 30 mg KW-3902, as a combined total (“active”) or placebo (“placebo”) falling into the three different categories designated for the primary endpoint of the study: Modified Success, Unchanged, and Failure, as described in Example 1.

### DETAILED DESCRIPTION OF THE PREFERRED EMBODIMENTS

The present disclosure is based, in part, on a surprisingly good con-elation between a patient's BNP or NT-pro-BNP levels and successful treatment with an AA₁RA. In studies involving administration of the AA₁RA KW-3902 to patients having impaired or inadequate renal function, including patients with congestive heart failure, we found a significant correlation between levels of BNP (or NT-pro-BNP) and the success of treatment with the AA₁RA. As discussed in more detail below, this parameter was surprisingly effective in identifying those patients that were most likely to respond to the AA₁RA therapy.

Provided herein are methods of treating subjects with cardio and/or renal dysfunction such as congestive heart failure (CHF), renal impairment, or the combination of CHF and renal impairment, including any symptoms associated with those disorders. Some embodiments relate to methods of improving renal function in subjects. Other embodiments relate to methods of maintaining renal function in a subject. Still other methods relate to methods of restoring renal function in a subject. Yet other methods relate to treating heart failure in a subject with renal impairment. Still other methods relate to maintaining or restoring the diuretic effect of a non adenosine-modifying diuretic in a subject. Yet other methods relate to preventing or delaying the onset of renal impairment in individuals with CHF. Still other methods relate to methods of treating CHF. The methods described herein involve the administration of a therapeutically effective amount of KW-3902 or a pharmaceutically acceptable salt, ester, amide, metabolite, or prodrug thereof.

In certain embodiments, the subject may be a mammal. The mammal may be selected from the group consisting of mice, rats, rabbits, guinea pigs, dogs, cats, sheep, goats, cows, primates, such as monkeys, chimpanzees, and apes, and humans. In some embodiments, the subject is a human. The term “subject” can be used interchangeably with the terms “individual” and “patient” herein.

In the embodiments described herein, the subject can be identified as having brain natriuretic peptide (“BNP”) levels of at least about 250 pg/mL, at least about 260 pg/mL, at least about 270 pg/mL, at least about 280 pg/mL, at least about 290 pg/mL, at least about 300 pg/mL, at least about 310 pg/mL, at least about 320 pg/mL, at least about 330 pg/mL, at least about 340 pg/mL, at least about 350 pg/mL, at least about 360 pg/mL, at least about 370 pg/mL, at least about 380 pg/mL, at least about 390 pg/mL, at least about 400 pg/mL, at least about 410 pg/mL, at least about 420 pg/mL, at least about 430 pg/mL, at least about 440 pg/mL, at least about 450 pg/mL, at least about 460 pg/mL, at least about 470 pg/mL, at least about 480 pg/mL, at least about 490 pg/mL, at least about 500 pg/mL, at least about 510 pg/mL, at least about 520 pg/mL, at least about 530 pg/mL, at least about 540 pg/mL, at least about 550 pg/mL, at least about 560 pg/mL, at least about 570 pg/mL, at least about 580 pg/mL, at least about 590 pg/mL, at least 600 pg/mL, at least about 620 pg/mL, at least about 640 pg/mL, at least about 660 pg/mL, at least about 680 pg/mL, at least about 700 pg/mL or more, or any number in between. Preferably the subject is identified as having BNP levels of at least about 450 pg/mL, e.g. at least 475 pg/mL, and most preferably identified as having BNP levels of at least about 500 pg/mL.

In some embodiments, the subject can be identified as having N-terminal pro-brain natriuretic peptide (“NT-ProBNP”) levels of at least about 1000 pg/mL, at least about 1100 pg/mL, at least about 1200 pg/mL, at least about 1300 pg/mL, at least about 1400 pg/mL, at least about 1500 pg/mL, at least about 1600 pg/ml, at least about 1700 pg/mL, at least about 1800 pg/mL, at least about 1900 pg/mL, at least about 2000 pg/mL, at least about 2100 pg/mL, at least about 2200 pg/mL, at least about 2300 pg/mL, at least about 2400 pg/mL, at least about 2500 pg/mL, at least about 2600 pg/mL, at least about 2700 pg/mL, at least about 2800 pg/mL, at least about 2900 pg/mL, at least about 3000 pg/mL, or more, or any number in between. Preferably, the subject is identified as having NT-ProBNP levels of at least about 1500 pg/mL, e.g., at least about 1750 pg/mL, and most preferably at least about 2000 pg/mL.

BNP is a cardiac hormone produced from the cardiac myocytes as a prepro hormone that consists of 134 amino acids. The pre hormone is then clipped into a pro-hormone, and subsequently further modified and released into the blood as the fragmented protein N-terminal proBNP (NT-proBNP) and the active BNP hormone. Serum levels of both BNP and NT-proBNP are commonly used to diagnose and grade the severity of heart failure in individuals. The higher the level of BNP or NT-proBNP, the more severe the heart failure is likely to be.

Table 1 sets forth average levels of BNP in individuals with and without heart failure.

TABLE 1
People who do not have CHF

| | Under | | | | |
|---|---|---|---|---|---|
| | age 45 | Age 45-54 | Age 55-64 | Age 65-74 | Over 75 |
| Average | 14 | 20 | 26 | 31 | 64 |
| concentration | | | | | |
| (pg/mL) | | | | | |

People who do have CHF

| | All Heart | | | | |
|---|---|---|---|---|---|
| | Failure | | | | |
| | Classes | Class | Class | Class | Class |
| | Combined | I | II | III | IV |
| Average | 526 | 146 | 326 | 575 | 897 |
| concentration | | | | | |
| (pg/mL) | | | | | |

BNP and NT-ProBNP levels can be determined using routine clinical procedures. Several commercially available tests for the measurement of BNP and/or NT-ProBNP, e.g., TRIAGE® BNP test (BioSite, Inc., San Diego, Calif.).

In certain embodiments, the individual being treated by the methods of the present invention suffers from renal impairment, e.g., miled to severe renal impairment. In other embodiments, the individual does not suffer from renal impairment. It will be appreciated that any method known to those skilled in the art for measuring renal function can be used in the methods described herein. For example, serum creatinine levels, creatinine clearance, glomerular filtration rate (GFR) and renal plasma flow (RPF) can be used to assess renal function. For example, individuals suffering from renal impairment can exhibit a creatinine clearance rate of about 20 mL/min to about 80 mL/min, e.g., about 25 mL/min, about 30 mL/min, about 35 mL/min, about 40 mL/min, about 45 mL/min, about 50 mL/min, about 55 mL/min, about 60 mL/min, about 65 mL/min, about 70 mL/min, about 75 mL/min, about 80 mL/min, or more, or any number in between. In some embodiments, patients exhibit a GFR of less than about 80 mL/min, for example about 20 mL/min, 30 mL/min, 40 mL/min, 50 mL/min, 60 mL/min 70 mL/min or 75 mL/min, or any number in between.

Accordingly, in some embodiments, the patient exhibits mildly impaired renal function (e.g., a GFR of about 50 to about 80 mL/min). In some embodiments, the patient exhibits moderately impaired renal function (e.g., a GFR of about 30 mL/min to about 50 mL/min). In yet other embodiments, the patient exhibits severely impaired renal function (e.g., a GFR of about 0 mL/min to about 30 mL/min). Individuals with impaired renal function can include individuals who suffer from heart failure, such as congestive heart failure, or other maladies that result in fluid overload, without having yet disrupted normal kidney function.

In some embodiments, the individual being treated by the methods disclosed herein have congestive heart failure. Congestive heart failure (CHF) is a condition in which impaired heart function exists. The impaired heart function can be accompanied by a build-up of body fluid. CHF often occurs when cardiac output is insufficient to meet metabolic demands of the body, or when the heart cannot meet the demands of operating at increased levels of filling/diastolic pressure. In some embodiments, the individual being treated by the methods disclosed herein have stable congestive heart failure. As used herein, the term “stable congestive heart failure” or “chronic congestive heart-failure” is given its ordinary meaning. For example, an individual with stable or chronic congestive heart failure can refer to an individual who has a documented history of congestive heart failure, including at least one prior symptom. Non-limiting examples of symptoms of congestive heart failure include dyspnea on exertion or at rest, orthopnea, paroxysmal nocturnal dyspnea, abdominal swelling, and peripheral edema. Stable congestive heart failure can also describe individuals with one prior sign of heart failure. Non-limiting examples of signs of congestive heart failure include jugular venous distension, ventricular gallop, rales, hepatomegaly, ascites or peripheral edema. Stable congestive heart failure can further refer to a current absence of excessive congestion, e.g., no or little ascites, and only mild basilar pulmonary rales and peripheral edema.

In some embodiments, individuals identified in the methods provided herein have acute congestive heart failure. Patients presenting with acute decompensated CHF can have an acute injury to the heart, such as a myocardial infarction, mitral regurgitation or ventricular septal rupture. Typically, the injury compromises myocardial performance (for example, a myocardial infarction) or valvular/chamber integrity (for example, mitral regurgitation or ventricular septal rupture). Such injuries can result in an acute rise in the left ventricular (LV) filing pressures. The rise in the LV filing pressures results in pulmonary edema and dyspnea. In some cases the acute CHF is due to an acute increase in systemic vascular resistance or volume overload secondary to medication non-compliance or dietary indiscretion.

In some embodiments, the individual being treated by the methods disclosed herein have acute fluid overload. In some embodiments, the individual has CHF and acute fluid overload. In other embodiments, the individual does not have CHF, but has acute fluid overload. In some embodiments, the patients presenting with acute fluid overload are in need of intravenous diuretic treatment. In some embodiments, individuals with acute fluid overload are in need of short-term hospitalization, and/or in need of intravenous diuretic therapy to treat the fluid overload. Patients with acute fluid overload can be identified using standard clinical diagnostic procedures. Non-limiting factors that are commonly evaluated in determining whether an individual requires hospitalization for acute fluid overload include pitting edema (2+) of lower extremities; jugular venous distension; pulmonary edema or pleural effusion; ascites; paroxysmal nocturnal dyspnea or 2-pillow orthopnea.

In some embodiments the subjects are in need of intravenous diuretic therapy. Patients in need of intravenous diuretic treatment can be identified using conventional diagnostic methods. For example, an individual in need of IV diuretic treatment can refer to an individual exhibiting one or more signs or symptoms of CHF, e.g., congestion of the lungs, liver, intestines and peripheral compartments, shortness of breath (dyspnea) fatigue, orthopnea, rales, pitting edema, elevated central venous pressure, pulmonary congestion, weight gain, volume overload, and elevated filling pressures, that cannot be managed taking oral therapy, such as oral dosage forms of diuretics.

In some embodiments, the individual being treated by the methods of the present invention is refractory to standard diuretic therapy. In other embodiments, the individual is not refractory to standard diuretic therapy.

The subjects identified in the methods described herein can be administered a therapeutically effective amount of an AA₁RA, e.g., KW-3902, or a pharmaceutically acceptable salt, ester, amide, metabolite, or prodrug thereof. As used herein, the term “therapeutically effective amount” refers to that amount of a composition being administered which will relieve to some extent one or more of the signs or symptoms of the condition being treated. Fore example, in some embodiments, the subject is provided a composition comprising 2.5 mg, 5 mg, 10 mg, 15, mg, 20 mg, 22 mg, 24 mg, 26 mg, 28 mg, 30 mg, 32 mg, 34 mg, 36 mg, 38 mg, 40 mg, 42 mg, 44 mg, 46 mg, 48 mg, 50 mg, 52 mg, 54 mg, 56 mg, 58 mg, 60 mg, 62 mg, 64 mg, 66 mg, 68 mg, 70 mg, 72 mg, 74 mg, 76 mg, 78 mg, 80 mg, 85 mg, 90 mg, 100 mg, or more, or any amount in between, of an AA₁RA, e.g., KW-3902 or a pharmaceutically acceptable salt, ester, amide, metabolite, or prodrug thereof. Preferably, the subject is provided a composition comprising between about 20 mg to about 40 mg KW-3902 or a pharmaceutically acceptable salt, ester, amide, metabolite, or prodrug thereof, e.g., 30 mg KW 3902.

KW-3902 is a xanthine-derived adenosine A₁ receptor antagonist (AA₁RA). Its chemical name is 8-(3-noradamantyl)-1,3-dipropylxanthine, also known as 3,7-dihydro-1,3-dipropyl-8-(3-tricyclo[3.3.1.0^{3,7}]nonyl)-1H-purine-2,6-dione, and its structure is

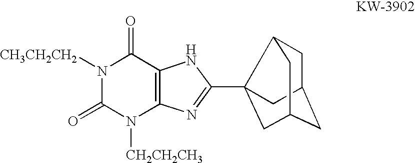

KW-3902 and related compounds useful in the practice of the present invention are described, for example, in U.S. Pat. Nos. 5,290,782, 5,395,836, 5,446,046, 5,631,260, 5,736,528, 6,210,687, and 6,254,889, the entire disclosure of all of which are hereby incorporated by reference herein, including any drawings.

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like.

The term “ester” refers to a chemical moiety with formula —(R)ₙ—COOR′, where R and R′ are independently selected from the group consisting of alkyl, cycloalkyl, aryl, heteroaryl (bonded through a ring carbon) and heteroalicyclic (bonded through a ring carbon), and where n is 0 or 1.

An “amide” is a chemical moiety with formula —(R)ₙ—C(O)NHR′ or —(R)ₙ—NHC(O)R′, where R and R′ are independently selected from the group consisting of alkyl, cycloalkyl, aryl, heteroaryl (bonded through a ring carbon) and heteroalicyclic (bonded through a ring carbon), and where n is 0 or 1. An amide may be an amino acid or a peptide molecule attached to a molecule of the present invention, thereby forming a prodrug.

The term “metabolite” refers to a compound to which KW-3902 is converted within the cells of a mammal. The pharmaceutical compositions of the present invention may include a metabolite of KW-3902 instead of KW-3902. The scope of the methods of the present invention includes those instances where KW-3902 is administered to the patient, yet the metabolite is the bioactive entity.

Some of the metabolites of KW-3902 are known. These include compounds where the propyl groups on the xanthine entity are hydroxylated, or that the propyl group is an acetylmethyl (CH₃C(O)CH₂—) group. Other metabolites include those in which the noradamantyl group is hydroxylated (i.e., is substituted with a —OH group) or oxylated (i.e., is substituted with a ═O group). Thus, examples of metabolites of KW-3902 include, but are not limited to, 8-(trans-9-hydroxy-3-tricyclo[3.3.1.0^{3,7}]nonyl)-1,3-dipropylxanthine (also referred to herein as “M1-trans”), S-(cis-9-hydroxy-3-tricyclo[3.3.1.0^{3,7}]nonyl)-1,3-dipropylxanthine (also referred to herein as “M1-cis”), 8-(trans-9-hydroxy-3-tricyclo[3.3.1.0^{3,7}]nonyl)-1-(2-oxopropyl)-3-propylxanthine and 1-(2-hydroxypropyl)-8-(trans-9-hydroxy-3-tricyclo[3.3.1.0^{3,7}]nonyl)-3-propylxanthine.

Any amine, hydroxy, or carboxyl side chain on the metabolites, esters, or amides of the above compounds can be esterified or amidified. The procedures and specific groups to be used to achieve this end is known to those of skill in the art and can readily be found in reference sources such as Greene and Wuts, Protective Groups in Organic Synthesis, 3^{rd} Ed., John Wiley & Sons, New York, N.Y., 1999, which is incorporated herein in its entirety.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

Adenosine A₁ receptor antagonists other than KW-3902 and its derivatives have been described in the art. For example, BG 9719 is described in U.S. Patent Application Publication No. 2002/0115687 A1. BG 9719 is also a xanthine-derivative compound, whose structure bears some similarity to that of KW-3902. However, the present inventors have surprisingly discovered that despite the structural similarity of these compounds, they behave remarkably differently in various ways.

Nevertheless, based on other similarities within the class of compounds, we believe that the data disclosed herein for KW-3902 are generalizable to the class of AA₁RAs, particularly with respect to the selection of patients most likely to benefit from the various AA₁RA therapies disclosed herein. Thus, even for AA₁RAs that do not have the same therapeutic benefits and desirable properties as KW-3902, patients most likely to benefit from an AA₁RA therapy can be identified by reference to their levels of BNP or NT-pro-BNP. Thus, in those instances where methods are disclosed herein in connection with KW-3902, we also contemplate that other AA₁RAs can be substituted therefor. Nonlimiting examples of such other AA₁RAs include, for example, compounds such as 1,3-dipropyl-8-{3-oxatricyclo[3.1.2.0.^{2,4}]oct-6(7)-yl}xanthine (also known as 1,3-dipropyl-8-[5,6-exo-epoxy-2(S)norbornyl]xanthine, ENX, CVT-124, and BG9719), 8-(3-noradamantyl)-1,3-dipropylxanthine (also known as KW-3902), theophyllilne, and caffeine. Other AA₁RAs are disclosed in U.S. Pat. Nos. 5,446,046, 5,631,260, and 5,668,139, the specification of all of which is hereby incorporated by reference herein in their entirety, including any drawings. The scope of the present invention includes all those AA₁RAs now known and all those AA₁RAs to be discovered in the future.

In some embodiments, the AA₁RA, e.g., KW-3902 or a pharmaceutically acceptable salt, ester, amide, metabolite, or prodrug thereof, can be administered parenterally. For example, intramuscularly, subcutaneously, intravenously, via intramedullary injections, as well as intrathecal, direct intraventricular, intraperitoneal, intranasal, or intraocular injections or the like. Preferably, the AA₁RA, e.g., KW-3902 or a pharmaceutically acceptable salt, ester, amide, metabolite, or prodrug thereof can provided intravenously in a continuous infusion.

In some embodiments, an AA₁RA, e.g., KW-3902 or a pharmaceutically acceptable salt, ester, amide, metabolite, or prodrug thereof, is provided in a single dose during the administration. For example, in some embodiments, about 20 mg, 30 mg, 40 mg or more of an AA₁RA, e.g., KW-3902 or a pharmaceutically acceptable salt, ester, amide, metabolite, or prodrug thereof, can be provided in a single dose, for example in a continuous intravenous infusion. In some embodiments, an AA₁RA, e.g., KW-3902 or a pharmaceutically acceptable salt, ester, amide, metabolite, or prodrug thereof is provided in more than one dose during the administration, for example, two, three or more doses of an AA₁RA, e.g., KW-3902 or a pharmaceutically acceptable salt, ester, amide, metabolite, or prodrug thereof can be provided in a single continuous intravenous infusion. Accordingly, in some embodiments, a dose of about 10 mg of an AA₁RA, e.g., KW-3902 or a pharmaceutically acceptable salt, ester, amide, metabolite, or prodrug thereof can be provided followed by a dose of about 15 mg or 20 mg in a continuous infusion. Alternatively, a dose of about 15 mg or 20 mg of an AA₁RA, e.g., KW-3902 or a pharmaceutically acceptable salt, ester, amide, metabolite, or prodrug thereof can be provided, followed by a dose of about 10 mg or 15 mg of the AA₁RA in a continuous infusion, and the like.

In some embodiments, the AA₁RA, e.g., KW-3902 or a pharmaceutically acceptable salt, ester, amide, metabolite, or prodrug thereof can be provided in a continuous infusion for a period of time of about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, or more. Preferably, the AA₁RA, e.g., KW-3902 or a pharmaceutically acceptable salt, ester, amide, metabolite, or prodrug thereof can be provided in a continuous infusion for a period of time of about 3 hours, 3.5 hours, 4 hours, 4.5 hours, or 5 hours, 5.5 hours, 6 hours, or 6.5 hours, or any amount of time in between. In some embodiments, a single dose of the an AA₁RA, e.g., KW-3902 or a pharmaceutically acceptable salt, ester, amide, metabolite, or prodrug thereof is provided in a continuous infusion over a period of about 3 hours, 3.5 hours, 4 hours or 4.5 hours, preferably about 4 hours. In some embodiments, two doses of the AA₁RA, e.g., KW-3902 or a pharmaceutically acceptable salt, ester, amide, metabolite, or prodrug thereof can be provided in a continuous infusion over a period of about 4 hours, 4.5 hours, 5 hours, 5.5 hours, 6 hours, 6.5 hours, or 7 hours. Optionally, the first dose can be provided over about 1.5 to about 2.5 hours, preferably 2 hours, and the second dose can be provided over about 3.5 hours, 4 hours, or 4.5 hours, preferably about 4 hours.

Thus, in some embodiments, an individual receiving chronic diuretic therapy is administered doses of a therapeutically effective amount of AA₁RA about every four days to about every month. Accordingly, in some embodiments, the AA₁RA can be administered to the individual receiving chronic diuretic therapy at least about every 4 days, about every 5 days, about every 6 days, about every 7 days, about every 8 days, about every 9 days, about every 10 days, about every 11 days, about every 12 days, about every 13 days, about every 14 days, about every 15 days, about every 16 days, about every 17 days, about every 18 days, about every 19 days, about every 20 days, about every 21 days, about every 22 days, about every 23 days, about every 24 days, about every 25 days, about every 26 days, about every 27 days, about every 28 days, about every 29 days, about every 30 days, about every 31 days, about every 40 days, about every 50 days, or about every 60 days, or any number of days in between.

In some embodiments, the AA₁RA, e.g., KW-3902 is administered daily, twice a day, three times a day, four times a day, five times a day, six times a day, or more. As stated above, the AA₁RA, e.g., KW-3902 or pharmaceutically acceptable salts, ester, amides, metabolites or prodrugs thereof can be administered orally. Optionally, the oral formulation of KW-3902 can provide for controlled release or sustained release of the active pharmaceutical ingredient, e.g., KW-302. Oral formulations of KW-3902 including controlled release formulation can be generated using routine methods known to those of skill in the art. A description of carrier materials useful in the oral formulations described herein can be found in the Remington: The Science and Practice of Pharmacy (20^{th} ed, Lippincott Williams & Wilkens Publishers (2003)), which is incorporated herein by reference in its entirety.

Combinations of AA₁RAs with Other Therapeutics

In some embodiments provided herein, the subject to be treated by the methods described herein can be administered an AA₁RA, e.g., KW-3902 or a pharmaceutically acceptable salt, ester, amide, metabolite or prodrug thereof in combination with another compound or therapeutic such as a non adenosine-modifying diuretic, an ACE, an ARB, a beta blocker, an aldosterone inhibitor or other compound or any combination thereof. In some embodiments, the administering step comprises administering said non adenosine-modifying diuretic, or other therapeutic (e.g., ACE, ARB, beta blocker, an aldosterone inhibitor and the like) and said an AA₁RA, e.g., KW-3902 or a pharmaceutically acceptable salt, ester, amide, metabolite or prodrug thereof nearly simultaneously. These embodiments include those in which the AA₁RA, e.g., KW-3902 or a pharmaceutically acceptable salt, ester, amide, metabolite or prodrug thereof and the non adenosine-modifying diuretic, or other therapeutic (e.g., ACE, ARB, beta blocker, an aldosterone inhibitor and the like) are in the same administrable composition, i.e., a single tablet, pill, or capsule, or a single solution for intravenous injection, or a single drinkable solution, or a single dragee formulation or patch, contains both compounds. The embodiments also include those in which each compound is in a separate administrable composition, but the subject is directed to take the separate compositions nearly simultaneously, i.e., one pill is taken right after the other or that one injection of one compound is made right after the injection of another compound, etc.

In other embodiments the administering step comprises administering the non adenosine-modifying diuretic, or other therapeutic (e.g., ACE, ARB, beta blocker, an aldosterone inhibitor and the like) first and then administering an AA₁RA, e.g., KW-3902 or a pharmaceutically acceptable salt, ester, amide, metabolite or prodrug thereof. In yet other embodiments, the administering step comprises administering an AA₁RA, e.g., KW-3902 or a pharmaceutically acceptable salt, ester, amide, metabolite or prodrug thereof, first, and then administering the non adenosine-modifying diuretic, or other therapeutic (e.g., ACE, ARB, beta blocker, an aldosterone inhibitor and the like). In these embodiments, the subject may be administered a composition comprising one of the compounds and then at some time, a few minutes or a few hours, later be administered another composition comprising the other one of the compounds. Also included in these embodiments are those in which the subject is administered a composition comprising one of the compounds on a routine or continuous basis while receiving a composition comprising the other compound occasionally.

Some embodiments provided herein provide for the administration of an AA₁RA, e.g., KW-3902 or a pharmaceutically acceptable salt, ester, amide, metabolite or prodrug thereof as described above and a non-adenosine modifying diuretic. In some embodiments, the non-adenosine modifying diuretic is a proximal diuretic, i.e., a diuretic that principally acts on the proximal tubule. Examples of proximal diuretics include, but are not limited to, acetazolamide, methazolamide, and dichlorphenamide. Carbonic anhydrase inhibitors are known to be diuretics that act on the proximal tubule, and are therefore, proximal diuretics. Thus, some embodiments provide compositions that include the combination of KW-3902 or a pharmaceutically acceptable salt, ester, amide, metabolite or prodrug thereof with a carbonic anhydrase inhibitor. Combinations of an AA₁RA (e.g., KW-3902), with any proximal diuretic now known or later discovered are within the scope of the embodiments disclosed herein.

In other embodiments, the non-adenosine modifying diuretic is a loop diuretic, i.e., a diuretic that principally acts on the loop of Henle. Examples of loop diuretics include, but are not limited to, furosemide (LASIX®), bumetanide (BUMEX®), and torsemide (TOREM®). Combinations of an AA₁RA, e.g., KW-3902 or a pharmaceutically acceptable salt, ester, amide, metabolite or prodrug thereof with any loop diuretic now known or later discovered are within the scope of the embodiments disclosed herein. In certain embodiments, the non adenosine-modifying diuretic used in the methods of the present invention is furosemide. In some embodiments, furosemide is administered in a dose of 20 mg, 40 mg, 60 mg, 80 mg, 100 mg, 120 mg, 140 mg, or 160 mg, or higher. The administration may be oral or intravenous. When furosemide is administered intravenously, it may be administered as a single injection or as a continuous infusion. When the administration is through a continuous infusion, the dosage of furosemide may be less than 1 mg per hour, 1 mg per hour, 3 mg per hour, 5 mg per hour, 10 mg per hour, 15 mg per hour, 20 mg per hour, 40 mg per hour, 60 mg per hour, 80 mg per hour, 100 mg per hour, 120 mg per hour, 140 mg per hour, or 160 mg per hour, or higher.

In yet other embodiments, the non-adenosine modifying diuretic is a distal diuretic, i.e., a diuretic that principally acts on the distal nephron. Examples of distal diuretics include, but are not limited to, metolazone, thiazides and amiloride. Combinations of KW-3902 or a pharmaceutically acceptable salt, ester, amide, metabolite or prodrug thereof with any distal diuretic now known or later discovered are within the scope of the embodiments disclosed herein.

In some embodiments, the subject can be administered an AA₁RA, e.g., KW-3902 or a pharmaceutically acceptable salt, ester, amide, metabolite or prodrug thereof and a beta-blocker. A number of beta-blockers are commercially available. These compounds include, but are not limited to, acebutolol hydrochloride, atenolol, betaxolol hydrochloride, bisoprolol fumarate, carteolol hydrochloride, esmolol hydrochloride, metoprolol, metoprolol tartrate, nadolol, penbutolol sulfate, pindolol, propranolol hydrochloride, succinate, and timolol maleate. Beta-blockers, generally, are beta₁ and/or beta₂ adrenergic receptor blocking agents, which decrease the positive chronotropic, positive inotropic, bronchodilator, and vasodilator responses caused by beta-adrenergic receptor agonists. The embodiments described herein include all beta-blockers now known and all beta-blockers discovered in the future.

In some embodiments provided herein, a subject is administered an AA₁RA, e.g., KW-3902 or a pharmaceutically acceptable salt, ester, amide, metabolite or prodrug thereof and an angiotensin converting enzyme inhibitor or an angiotensin II receptor blocker. A number of ACE inhibitors are commercially available. These compounds, whose chemical structure is somewhat similar, include lisinopril, enalapril, quinapril, ramipril, benazepril, captopril, fosinopril, moexipril, trandolapril, and perindopril. ACE inhibitors, generally, are compounds that inhibit the action of angiotensin converting enzyme, which converts angiotensin I to angiotensin II. The embodiments described herein include all ACE inhibitors now known and all ACE inhibitors discovered in the future.

A number of ARBs are also commercially available or known in the art. These compounds include losartan, irbesartan, candesartan, telmisartan, eposartan, and valsartan. ARBs reduce blood pressure by relaxing blood vessels. This allows better blood flow. ARBs function stems from their ability to block the binding of angiotensin II, which would normally cause vessels to constrict. The embodiments disclosed herein include all ARBs now known and all ARBs discovered in the future.

In some embodiments provided herein, a subject is administered an AA₁RA, e.g., KW-3902 or a pharmaceutically acceptable salt, ester, amide, metabolite, or prodrug thereof and an aldosterone inhibitor. A number of aldosterone inhibitors are commercially available. These compounds include, but are not limited to, spironolactone (ALDACTON®) and eplerenone (INSPRA®). The embodiments disclosed herein include all aldosterone inhibitors now known and all aldosterone inhibitors discovered in the future.

In still other embodiments, the subject can be administered an AA₁RA, e.g., KW-3902 or a pharmaceutically acceptable salt, ester, amide, metabolite or prodrug thereof and an a prophylactically or therapeutically effective amount of an anticonvulsant. Several anticonvulsants are known in the art and are useful in the compositions and methods described herein. See, e.g., U.S. Patent Application Publication No. 2005/0070524 Extensive listings of anticonvulsants can also be found, e.g., in Goodman and Gilman's “The Pharmaceutical Basis Of Therapeutics”, 8th ed., McGraw-Hill, Inc. (1990), pp. 436-462, and “Remington's Pharmaceutical Sciences”, 17th ed., Mack Publishing Company (1985), pp. 1075-1083, the disclosures of which are hereby expressly incorporated by reference in their entireties. Non-limiting examples of anticonvulsants that can be used in the compositions and methods disclosed hererin include diazepam, midazolam, phenyloin, pheonobarbital, mysoline, clonazepam, clorazepate, carbamazepine, oxcarbazepine, valproic acid, valproate, gabapentin, topiramate, felbamate, tiagabine, lamotrigine, famotodine, mephenyloin, ethotoin, mephobarbital, ethosuximide, methsuximide, phensuximide, trimethadione, paramethadione, phenacemide, acetazolamide, progabide, divalproex sodium, metharbital, clobazam, sulthiame, diphenylan, levetriacetam, primidone, lorazepam, thiopentione, propofol, and zonisamide, or a pharmaceutically acceptable salt, prodrug, ester, or amide thereof. However, the inclusion of other anticonvulsants, now known or discovered in the future, is within the scope of the present invention.

In some embodiments, an anticonvulsant can be provided in such amount as will be therapeutically or prophylactically effective in the treatment or control of seizures. It will be appreciated that the amount of anticonvulsant contained in an individual dose of each dosage form of the compositions need not in itself constitute an effective prophylactic amount, as the necessary effective amount could be reached by administration of a number of individual doses. Those skilled in the art will appreciate that the amount of anticonvulsant agent present in the compositions and administered to individuals disclosed herein will vary depending upon the age, sex, and bodyweight of the subject to be treated, the particular method and scheduling of administration, and what other anticonvulsant agent, if any is present in the compositions disclosed herein or administered in the methods disclosed herein. Dosage amounts for an individual patient may thus be above or below the typical dosage ranges. Generally speaking, the anticonvulsant agent can be employed in any amount known to be effective at treating, preventing or controlling seizures. The doses may be single doses or multiple doses per day, with the number of doses taken per day and the time allowed between doses varying depending on the individual needs of the patient. Optimization of treatment, including dosage amount, method and time of administration can be routinely determined by the skilled practitioner. Specific dosage levels for anticonvulsants that can be used in the pharmaceutical compositions and methods described herein, are included, for example, in the “Physicians' Desk Reference”, 2003 Edition (Medical Economics Data Production Company, Montvale, N.J.) as well as in other reference works including Goodman and Gilman's “The Pharmaceutical Basis of Therapeutics” and “Remington's Pharmaceutical Sciences,” the disclosures of which are all hereby expressly incorporated by reference. Representative examples of dosage ranges of anticonvulsants are described below, however, it should be noted that the dosage ranges given below indicate only the typical dosage amounts administered to patients for that particular anticonvulsant agent for the treatment of seizures or epilepsy. Thus they should not be construed as limiting amounts for the purpose of the present invention, as actual therapeutically effective dosage amounts for a patient may be more or less than the exemplary dosage range, depending on the individual.

### Methods of Treatment

A significant problem encountered in treating certain conditions with individual medications is that following a course of therapy the patients become refractory to the treatment, and begin to respond less and less to the medication until they do not respond at all. This problem is very common in patients who suffer from, for example, congestive heart failure, and are treated with diuretics.

Individual diuretics act on a specific segment of nephrons, e.g., proximal tubule, loop of Henle, or distal tubule. One mechanism by which diuretics increase urine volume is that they inhibit reabsorption of sodium and accompanying water passing through the nephron. Thus, for example, a loop diuretic inhibits reabsorption in the loop of Henle. As a consequence, higher concentrations of sodium are passed downstream to the distal tubule. This initially results in a greater volume of urine, hence the diuretic effect. However, the distal portion of the tubule recognizes the increase in sodium concentration and the kidney reacts in two ways; one is to increase sodium reabsorption elsewhere in the nephron; the other is to feedback via adenosine A₁ receptors to the afferent arteriole where vasoconstriction occurs. This feedback mechanism is known as tubuloglomerular feedback (TGF). This vasoconstriction results in decreased renal blood flow and decreased glomerular filtration rate (GFR). With time, these two mechanisms result in a decrease in diuretic effect and worsening of renal function. This sequence of events contributes to the progression of disease.

The present inventors have made the surprising discovery that KW-3902 exhibits pronounced therapeutic benefits in subjects with BNP levels above about 500 pg/ml compared to subjects with BNP levels above, for example 250 pg/ml. Unexpectedly, a higher percentage of subjects with acute CHF and mild to several renal impairment reported improvements in dyspnea and were changed from IV to oral diuretic therapy following treatment with KW-3902 in a group of individuals identified with BNP levels above 500 pg/ml at the start of treatment, compared to a group of individuals with BNP levels above 250 pg/ml. Accordingly, methods described herein are directed to treatment regiments for the CHF and/or renal impairment in subjects with BNP levels above about 450 pg/ml, e.g., above about 460 pg/mL, above about 470 pg/mL, above about 480 pg/mL, above about 500 pg/mL or more, or any number in between and/or subjects with NT-proBNP levels above about 1500 pg/ml, e.g., above about 1600 pg/mL, above about 1700 pg/mL, above about 1800 pg/mL, above about 1900 pg/mL, above about 2000 pg/mL or more, or any number in between.

The present inventors have also discovered that the combination of an AA₁RA, e.g., KW-3902 with a standard diuretic is beneficial to patients who are refractory to standard therapy. KW-3902 also blocks the TGF mechanism mediated by adenosine (via A₁ receptors) described above. This ultimately allows for increased GFR and improved renal function, which ultimately results in more fluid passing through the loop of Henle and the distal tubule. In addition, KW-3902 inhibits the reabsorption of sodium (and, therefore, water) in the proximal tubule, which results in diuresis. Furthermore, KW-3902 is an inhibitor of TGF, which can counteract the adverse effect of some diuretics, such as proximal diuretics, which activate or promote TGF.

The combinations of AA₁RAs, e.g., KW-3902 and non adenosine-modifying diuretics described herein act synergistically to further improve renal function for continued diuresis. In addition, most CHF patients are also on additional diuretics. The combination allows for greater efficacy of other more distally acting diuretics by improving renal blood flow, renal function, and in some cases, drug delivery.

Thus, some embodiments relate to methods of treating an subject with impaired renal function, comprising identifying a subject with impaired renal function and BNP levels above about 450 pg/ml, e.g., above about 460 pg/mL, above about 470 pg/mL, above about 480 pg/mL, above about 500 pg/mL or more, or any number in between and/or subjects with NT-proBNP levels above about 1500 pg/ml, e.g., above about 1600 pg/mL, above about 1700 pg/mL, above about 1800 pg/mL, above about 1900 pg/mL, above about 2000 pg/mL or more, or any number in between, and administering to the patient a therapeutically effective amount of an AA₁RA, e.g., KW-3902 or a pharmaceutically acceptable salt, ester, amide, metabolite or prodrug thereof. Optionally, in some embodiments, the methods include the step of administering a non-adenosine modifying diuretic to the subject.

Some embodiments relate to a methods of inducing a diuretic effect in a subject comprising identifying a subject with BNP levels above about 450 pg/ml, e.g., above about 460 pg/mL, above about 470 pg/mL, above about 480 pg/mL, above about 500 pg/mL or more, or any number in between and/or subjects with NT-proBNP levels above about 1500 pg/ml, e.g., above about 1600 pg/mL, above about 1700 pg/mL, above about 1800 pg/mL, above about 1900 pg/mL, above about 2000 pg/mL or more, or any number in between, and administering to the patient a therapeutically effective amount of an AA₁RA, e.g., KW-3902 or a pharmaceutically acceptable salt, ester, amide, metabolite or prodrug thereof in combination with a second pharmaceutical composition capable of inducing a diuretic effect. In some embodiments, the subject is refractory to standard diuretic therapy.

Other embodiments relate to a method of maintaining or restoring the diuretic effect of a non-adenosine modifying diuretic in a patient comprising identifying a subject with BNP levels above about 450 pg/ml, e.g., above about 460 pg/mL, above about 470 pg/mL, above about 480 pg/mL, above about 500 pg/mL or more, or any number in between and/or subjects with NT-proBNP levels above about 1500 pg/ml, e.g., above about 1600 pg/mL, above about 1700 pg/mL, above about 1800 pg/mL, above about 1900 pg/mL, above about 2000 pg/mL or more, or any number in between, and administering to the patient a therapeutically effective amount of an AA₁RA, e.g., KW-3902 or a pharmaceutically acceptable salt, ester, amide, metabolite or prodrug thereof. Optionally, a non-adenosine modifying diuretic is also administered to the subject.

Other embodiments relate to a method of maintaining or restoring renal function in a subject comprising identifying a identifying a subject in need of maintenance or restoration of renal function with BNP levels above about 450 pg/ml, e.g., above about 460 pg/mL, above about 470 pg/mL, above about 480 pg/mL, above about 500 pg/mL or more, or any number in between and/or subjects with NT-proBNP levels above about 1500 pg/ml, e.g., above about 1600 pg/mL, above about 1700 pg/mL, above about 1800 pg/mL, above about 1900 pg/mL, above about 2000 pg/mL or more, or any number in between, and administering to the patient a therapeutically effective amount of an AA₁RA, e.g., KW-3902 or a pharmaceutically acceptable salt, ester, amide, metabolite or prodrug thereof. Optionally, the an AA₁RA, e.g., KW-3902 is administered with a second pharmaceutical composition capable of inducing a diuretic effect.

Still other embodiments relate to a method of maintaining or restoring renal function in a patient with CHF comprising identifying a patient with CHF with BNP levels above about 450 pg/ml, e.g., above about 460 pg/mL, above about 470 pg/mL, above about 480 pg/mL, above about 500 pg/mL or more, or any number in between and/or subjects with NT-proBNP levels above about 1500 pg/ml, e.g., above about 1600 pg/mL, above about 1700 pg/mL, above about 1800 pg/mL, above about 1900 pg/mL, above about 2000 pg/mL or more, or any number in between, and administering to the patient a therapeutically effective amount of an AA₁RA, e.g., KW-3902 or a pharmaceutically acceptable salt, ester, amide, metabolite or prodrug thereof. Optionally, the AA₁RA, e.g., KW-3902 or pharmaceutically acceptable salt, ester, amide, metabolite, or prodrug thereof is administered in combination with second pharmaceutical composition capable of inducing a diuretic effect.

In the context of the present disclosure, by “maintaining” renal function it is meant that the renal function, as measured by creatinine clearance rate, remains unchanged for a period of time after the start of the therapy. In other words, by “maintaining” renal function it is meant that the rate of renal impairment, i.e., the rate of decrease in the urine creatinine clearance rate, is slowed or arrested for a period of time, however brief that period may be. By “restoring” renal function it is meant that the renal function, as measured by urine creatinine clearance rate, has improved, i.e., has become higher, after the start of the treatment. In certain embodiments, the second pharmaceutical composition comprises a loop diuretic and a distal diuretic.

In a further aspect, the present invention relates to a method of treating a subject that is refractory to standard diuretic therapy, comprising identifying a patient refractory to standard diuretic therapy with BNP levels above about 450 pg/ml, e.g., above about 460 pg/mL, above about 470 pg/mL, above about 480 pg/mL, above about 500 pg/mL or more, or any number in between and/or subjects with NT-proBNP levels above about 1500 pg/ml, e.g., above about 1600 pg/mL, above about 1700 pg/mL, above about 1800 pg/mL, above about 1900 pg/mL, above about 2000 pg/mL or more, or any number in between, and administering to the patient a therapeutically effective amount of an AA₁RA, e.g., KW-3902 or a pharmaceutically acceptable salt, ester, amide, metabolite or prodrug thereof. Optionally, an AA₁RA, e.g., KW-3902 or pharmaceutically acceptable salt, ester, amide, metabolite, or prodrug thereof is administered in combination with second pharmaceutical composition capable of inducing a diuretic effect.

Certain patients who suffer from a cardiac condition, such as congestive heart failure, later develop renal impairment. The present inventors have discovered that if a patient presented with a cardiac condition, and little to no renal impairment, is treated with a pharmaceutical composition as described herein, the onset of renal impairment is delayed or arrested, compared to a patient who receives standard treatment. Thus, aspects of the present invention relate to a method of preventing the deterioration of renal function, delaying the onset of renal impairment, or arresting the progress of renal impairment in a patient comprising identifying a patient with CHF with BNP levels above about 450 pg/ml, e.g., above about 460 pg/mL, above about 470 pg/mL, above about 480 pg/mL, above about 500 pg/mL or more, or any number in between and/or subjects with NT-proBNP levels above about 1500 pg/ml, e.g., above about 1600 pg/mL, above about 1700 pg/mL, above about 1800 pg/mL, above about 1900 pg/mL, above about 2000 pg/mL or more, or any number in between, and administering to the patient a therapeutically effective amount of KW-3902 or a pharmaceutically acceptable salt, ester, amide, metabolite or prodrug thereof. Optionally, the AA₁RA, e.g., KW-3902 or pharmaceutically acceptable salt, ester, amide, metabolite, or prodrug thereof is administered in combination with second pharmaceutical composition capable of inducing a diuretic effect, such as a non-adenosine modifying diuretic.

The term “treating” or “treatment” does not necessarily mean total cure. Any alleviation of any undesired signs or symptoms of the disease to any extent or the slowing down of the progress of the disease can be considered treatment. Furthermore, treatment may include acts that may worsen the patients overall feeling of well being or appearance. Treatment may also include lengthening the life of the patient, even if the symptoms are not alleviated, the disease conditions are not ameliorated, or the patient's overall feeling of well being is not improved. Thus, in the context of the present invention, increasing the urine output volume, decreasing the level of serum creatinine, or increasing creatinine clearance, may be considered treatment, even if the patient is not cured or does not generally feel better.

In another aspect, the present invention relates to a method of treating a patient suffering from CHF comprising identifying a patient with BNP levels above about 450 pg/ml, e.g., above about 460 pg/mL, above about 470 pg/mL, above about 480 pg/mL, above about 500 pg/mL or more, or any number in between and/or subjects with NT-proBNP levels above about 1500 pg/ml, e.g., above about 1600 pg/mL, above about 1700 pg/mL, above about 1800 pg/mL, above about 1900 pg/mL, above about 2000 pg/mL or more, or any number in between, and administering to the patient a therapeutically effective amount of an AA₁RA, e.g., KW-3902 or a pharmaceutically acceptable salt, ester, amide, metabolite or prodrug thereof. Optionally, the AA₁RA, e.g., KW-3902 or pharmaceutically acceptable salt, ester, amide, metabolite, or prodrug thereof is administered in combination with second pharmaceutical composition capable of inducing a diuretic effect.

In a further aspect, the present invention relates to a method of improving overall health outcomes, decreasing morbidity rates, or decreasing mortality rates in patients comprising identifying a patient in need thereof, with BNP levels above about 450 pg/ml, e.g., above about 460 pg/mL, above about 470 pg/mL, above about 480 pg/mL, above about 500 pg/mL or more, or any number in between and/or subjects with NT-proBNP levels above about 1500 pg/ml, e.g., above about 1600 pg/mL, above about 1700 pg/mL, above about 1800 pg/mL, above about 1900 pg/mL, above about 2000 pg/mL or more, or any number in between, and administering to the patient a therapeutically effective amount of an AA₁RA, e.g., KW-3902 or a pharmaceutically acceptable salt, ester, amide, metabolite or prodrug thereof. Optionally, the AA₁RA, e.g., KW-3902 or pharmaceutically acceptable salt, ester, amide, metabolite, or prodrug thereof is administered in combination with second pharmaceutical composition capable of inducing a diuretic effect.

Overall health outcomes are determined by various means in the art. For example, improvements in morbidity and/or mortality rates, improvements in the patient's general feelings, improvements in the quality of life, improvements in the level of comfort at the end of life, and the like, are considered when overall health outcome are determined. Mortality rate is the number of patients who die while undergoing a particular treatment for a period of time compared to the overall number of patients undergoing the same or similar treatment over the same period of time. Morbidity rates are determined using various criteria, such as the frequency of hospital stays, the length of hospital stays, the frequency of visits to the doctor's office, the dosage of the medication being administered, and the like.

In some embodiments, the patient whose overall health outcome, morbidity and/or mortality rate is being improved suffers from CHF. In other embodiments, the patient suffers from renal impairment. In some embodiments, the patient suffers from CHF and renal impairment.

Also provided herein are methods of improving the treatment time to achieve adequate diuresis in an individual experiencing acute fluid overload. The method can include the steps of identifying an individual with BNP levels above about 450 pg/ml, e.g., above about 460 pg/mL, above about 470 pg/mL, above about 480 pg/mL, above about 500 pg/mL or more, or any number in between and/or subjects with NT-proBNP levels above about 1500 pg/ml, e.g., above about 1600 pg/mL, above about 1700 pg/mL, above about 1800 pg/mL, above about 1900 pg/mL, above about 2000 pg/mL or more, or any number in between, in need of hospitalization for intravenous diuretic treatment with hospitalizing the individual and administering to the individual intravenous diuretic therapy and a therapeutically effective amount of an AA₁RA, e.g., KW-3902, or a pharmaceutically acceptable salt, ester, amide, metabolite, or prodrug thereof. As used herein the term “adequate diuresis” refers to diuresis sufficient that the patient is no longer in need of intravenous diuretic therapy, as determined using conventional diagnostic methods.

In some embodiments, treatment with the AA₁RA, e.g., KW-3902 is effective to reduce the amount of diuretic therapy needed in the individual. In some embodiments, the daily dose of diuretic can be reduced by about 1 mg to about 160 mg. For example, the daily dose of diuretic can be reduced by at least about 1 mg/day, about 5 mg/day, about 10 mg/day, about 15 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, about 140 mg/day, about 150 mg/day, about 160 mg/day, about 200 mg/day, any number in between or more. Accordingly, in some embodiments, the diuretic is furosemide, and the daily dose of furosemide can be reduced by about 1 mg/day, about 5 mg/day, about 10 mg/day, about 15 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, about 140 mg/day, about 150 mg/day, about 160 mg/day, about 200 mg/day, or more.

In other embodiments, the methods described herein reduce the term that the individual is in need of intravenous diuretic therapy. Accordingly, in some embodiments the methods provided herein reduce the term that the individual is in need of intravenous diuretic therapy by at least about 4 hours, 6 hours, 12 hours 18 hours, 24 hours, 32 hours, 48 hours, 60 hours, 72 hours, 84 hours, 96 hours, or 108 hours, or any number of hours in between. In some embodiments, the amount of KW-3902 is effective to reduce the term of intravenous diuretic therapy by more than 108 hours. In preferred embodiments, the amount of KW-3902 is administered to the patient in individual daily doses of about 30 mg.

In some embodiments, the methods provided herein reduce the term of short term hospitalization by at least about 4 hours, 6 hours, 12 hours 18 hours, 24 hours, 32 hours, 48 hours, 60 hours, 72 hours, 84 hours, 96 hours, or 108 hours, or any number of hours in between. In some embodiments, the methods provided herein reduce the term of the short-term hospitalization by more than 100 hours.

In some embodiments, the amount of the AA₁RA, e.g., KW-3902 administered to the patient is effective to improve renal function. For example, in some embodiments, the amount of an AA₁RA, e.g., KW-3902 is effective to decrease serum creatinine levels by about 0.01 to about 2.0 mg/dL.

The present inventors have made the discovery that administration of an AA₁RA (e.g., KW-3902) provides for improved renal function in individuals receiving chronic diuretic therapy that persists over an unexpectedly long period of time.

As used herein, the phrase “chronic diuretic therapy” or variations thereof, e.g., “chronic diuretics” refers to continuous diuretic therapy (e.g., at least daily therapy) for a period of time. Individuals identified as receiving chronic diuretic therapy, therefore, can refer to individuals that have been taking daily diuretics continuously over at least about three weeks, at least about 4 weeks, at least about 6 weeks, at least about 10 weeks, or at least about 12 weeks, or more, or for any period of time in between. Continuation of chronic diuretic therapy refers to substantially uninterrupted daily diuretic therapy.

Some embodiments disclosed herein are intended to provide treatment for cardiovascular disease, which may include congestive heart failure, hypertension, asymptomatic left ventricular dysfunction, coronary artery disease, or acute myocardial infarction. The subject can have levels of BNP levels above about 450 pg/ml, e.g. above about 460 pg/mL, above about 470 pg/mL, above about 480 pg/mL, above about 500 pg/mL or more, or any number in between and/or subjects with NT-proBNP levels above about 1500 pg/ml, e.g., above about 1600 pg/mL, above about 1700 pg/mL, above about 1800 pg/mL, above about 1900 pg/mL, above about 2000 pg/mL or more, or any number in between. In some instances, subjects suffering from a cardiovascular disease are in need of after-load reduction. The methods disclosed herein are suitable to provide treatment for these subjects as well. Certain subjects who suffer from a cardiac condition, such as congestive heart failure, later develop renal impairment.

Some embodiments disclosed herein relate to the treatment of cardiovascular diseases using a combination of a beta-blocker, and an AA₁RA. The present inventors have discovered that the combination of AA₁RAs and beta blockers is beneficial in either congestive heart failure (CHF) or hypertension, or any of the other indications set forth herein. See, co-pending U.S. application Ser. No. 10/785,446 entitled “Method of Treatment of Disease Using and Adenosine A1 Receptor Antagonist,” filed Feb. 23, 2004, herein expressly incorporated by reference in its entirety.

Beta-blockers are known to have antihypertensive effects. While the exact mechanism of their action is unknown, possible mechanisms, such as reduction in cardiac output, reduction in plasma renin activity, and a central nervous system sympatholytic action, have been put forward. From various clinical studies, it is clear that administration of beta-blockers to subjects with hypertension results initially in a decrease in cardiac output, little immediate change in blood pressure, and an increase in calculated peripheral resistance. With continued administration, blood pressure decreases within a few days, cardiac output remains reduced, and peripheral resistance falls toward pretreatment levels. Plasma renin activity is also reduced markedly in subjects with hypertension, which will have an inhibitory action on the renin-angiotensin system, thus decreasing the after-load and allowing for more efficient forward function of the heart. The use of these compounds has been shown to increase survival rates among subjects suffering from CHF or hypertension. The compounds are now part of the standard of care for CHF and hypertension. The combination of an AA₁RA (e.g., KW-3902), a beta blocker, acts synergistically to further improve the condition of subjects with hypertension or CHF. The methods of administration provided herein can also reduce the potential of related adverse events occurring, such as seizures or convulsions. The diuretic effect of AA₁RAs, especially in salt-sensitive hypertensive subjects along with the blockage of beta adrenergic receptors decreases blood pressure through two different mechanisms, whose effects build on one another. In addition, most CHF subjects are also on additional diuretics. The combination allows for greater efficacy of other more distally acting diuretics by improving renal blood flow and renal function.

Beta-blockers are well established in the treatment of hypertension. The addition of AA₁RAs will further treat hypertension via its diuretic effect from inhibiting sodium reabsorption through the proximal tubule. In addition, since many hypertensive subjects are sodium sensitive, the addition of an AA₁RA to a beta-blocker will result in further blood pressure reduction. AA₁RA action on tubuloglomerular feedback further improves renal function to result in greater diuresis and lower blood pressure.

In another aspect, the invention relates to the treatment of renal and/or cardiac diseases using a combination of an AA₁RA administered as described above and an angiotensin converting enzyme (ACE) inhibitor or an angiotensin II receptor blocker (ARB). The subject to be treated can have BNP levels above about 450 pg/ml, e.g., above about 460 pg/mL, above about 470 pg/mL, above about 480 pg/mL, above about 500 pg/mL or more, or any number in between and/or subjects with NT-proBNP levels above about 1500 pg/ml, e.g., above about 1600 pg/mL, above about 1700 pg/mL, above about 1800 pg/mL, above about 1900 pg/mL, above about 2000 pg/mL or more, or any number in between. AA₁RAs, ACE inhibitors and ARBs have individually been shown to be somewhat effective in the treatment of cardiac disease, such as congestive heart failure, hypertension, asymptomatic left ventricular dysfunction, or acute myocardial infarction, or renal disease, such as diabetic nephropathy, contrast-mediated nephropathy, toxin-induced renal injury, or oxygen free-radical mediated nephropathy.

The present inventors have discovered that the combination of AA₁RAs and ACE inhibitors or ARBs is beneficial in either congestive heart failure (CHF) or hypertension. See, co-pending U.S. application Ser. No. 10/785,446. The use of ACE inhibitors and ARBs in CHF relies on inhibition of renin-angiotensin system. These compounds decrease the after-load, thereby allowing for more efficient forward function of the heart. In addition, renal function is “normalized” or improved such that subjects remove excess fluid more effectively. The use of these compounds has been shown to increase survival rates among subjects suffering from CHF or hypertension. The compounds are now part of the standard of care for CHF and hypertension.

The combination of AA₁RAs and ACE inhibitors or ARBs acts synergistically to further improve renal function for continued diuresis. In addition, most CHF subjects are also on additional diuretics. The combination allows for greater efficacy of other more distally acting diuretics by improving renal blood flow and renal function.

Both ACE inhibitors and ARBs are well established in the treatment of hypertension via their action through the renin-angiotensin system. The addition of AA₁RAs will further treat hypertension via its diuretic effect from inhibiting sodium reabsorption through the proximal tubule. In addition, since many hypertensive subjects are sodium sensitive, the addition of an AA₁RA to an ACE inhibitor or an ARB will result in further blood pressure reduction. AA₁RA action on tubuloglomerular feedback further improves renal function to result in greater diuresis and lower blood pressure.

ACE inhibitors and ARBs are also known to prevent some of the renal damage induced by the immunosuppresant, cyclosporin A. However, there is a renal damaging effect despite their use. The present inventors have discovered that the combination ACE inhibitors and ARBs with AA₁RAs would be more effective in preventing drug-induced nephrotoxicity, such as that induced by cyclosporin A, contrast medium (iodinated), and aminoglycoside antibiotics. In this setting there is renal vasoconstriction that can be minimized by both compounds. In addition, direct negative effects on the tubular epithelium by cyclosporin is less prominent in the setting of adenosine A₁ receptor antagonism, in that blocking A₁ receptors decreases active processes. Furthermore, there are fewer oxidative by-products that are injurious to the tubular epithelium. In addition, the inhibitory effect of AA₁RA blockade on the tubuloglomerular feedback mechanism helps preserve function in the setting of nephrotoxic drugs.

It is known that ACE inhibitors and ARBs are beneficial in preventing the worsening of renal dysfunction in diabetics as measured by albuminuria (proteinuria). Once diabetes begins, glucosuria develops and the kidneys begin to actively reabsorb glucose, especially through the proximal convoluted tubule. This active process may result in oxidative stress and begin the disease process of diabetic nephropathy. Early manifestations of this process are hypertrophy and hyperplasia of the kidney. Ultimately, the kidney begins to manifest other signs such as microalbuminuria and decreased function. It is postulated that the active reabsorption of glucose is mediated in part by adenosine A₁ receptors. Blockade of this process by an AA₁RA limits or prevents the early damage manifested in diabetics.

The combination of AA₁RA and ACE inhibitors or ARBs, as disclosed herein, works to limit both early and subsequent damage to the kidneys in diabetes. The presently disclosed combinations are given at the time of diagnosis of diabetes or as soon as glycosuria is detected in at risk subjects (metabolic syndrome). The long-term treatment using the combinations of the present invention includes daily administration of the pharmaceutical compositions described herein.

Other embodiments relate to a method of treating cardiovascular disease or renal disease comprising identifying a subject in need of such treatment with BNP levels above about 450 pg/ml, e.g., above about 460 pg/mL, above about 470 pg/mL, above about 480 pg/mL, above about 500 pg/mL or more, or any number in between and/or subjects with NT-proBNP levels above about 1500 pg/ml, e.g., above about 1600 pg/mL, above about 1700 pg/mL, above about 1800 pg/mL, above about 1900 pg/mL, above about 2000 pg/mL or more, or any number in between, and administering a combination of an AA₁RA as described above, and an angiotensin converting enzyme (ACE) inhibitor or an angiotensin II receptor blocker (ARB) to said subject. In certain embodiments, the subject may be a mammal.

The methods disclosed herein are intended to provide treatment for cardiovascular disease, which may include congestive heart failure, hypertension, asymptomatic left ventricular dysfunction, or acute myocardial infarction. The methods disclosed herein can reduce the risk of adverse side effects, such as convulsions or seizures. In some instances, subjects suffering from a cardiovascular disease are in need of after-load reduction. The methods disclosed herein are suitable to provide treatment for these subjects as well.

The methods disclosed herein are also intended to provide treatment for renal disease, which may include renal hypertrophy, renal hyperplasia, microproteinuria, proteinuria, diabetic nephropathy, contrast-mediated nephropathy, toxin-induced renal injury, or oxygen free-radical mediated nephropathyhypertensive nephropathy, diabetic nephropathy, contrast-mediated nephropathy, toxin-induced renal injury, or oxygen free-radical mediated nephropathy.

Still other embodiments relate to methods of treating alkosis in a subject with BNP levels above about 450 pg/ml, e.g., above about 460 pg/mL, above about 470 pg/mL, above about 480 pg/mL, above about 500 pg/mL or more, or any number in between and/or subjects with NT-proBNP levels above about 1500 pg/ml, e.g., above about 1600 pg/mL, above about 1700 pg/mL, above about 1800 pg/mL, above about 1900 pg/mL, above about 2000 pg/mL or more, or any number in between, by administering to said subject an AA₁RA, e.g. KW-3902. Alkalosis is an acid-base disturbance caused by an elevation in plasma bicarbonate (HCO₃⁻) concentration. It is a primary pathophysiologic event characterized by the gain of bicarbonate or the loss of nonvolatile acid from extracellular fluid. The kidney preserves normal acid-base balance by two mechanisms: bicarbonate reclamation, mainly in the proximal tubule, and bicarbonate generation, predominantly in the distal nephron. Bicarbonate reclamation is mediated mainly by a Na⁺—H⁺ antiporter and to a smaller extent by the H⁺-ATPase (adenosine triphosphatase). The principal factors affecting HCO₃⁻ reabsorption include effective arterial blood volume, glomerular filtration rate, potassium, and partial pressure of carbon dioxide. Bicarbonate regeneration is primarily affected by distal Na⁺ delivery and reabsorption, aldosterone, systemic pH, ammonium excretion, and excretion of titratable acid.

There are a number of different types of alkalosis, for instance metabolic alkalosis and respiratory alkalosis. Respiratory alkalosis is a condition that affects mountain climbers in high altitude situations.

To generate metabolic alkalosis, either a gain of base or a loss of acid must occur. The loss of acid may be via the upper gastrointestinal tract or via the kidney. Excess base may be gained by oral or parenteral HCO₃⁻ administration or by lactate, acetate, or citrate administration.

Factors that help maintain metabolic alkalosis include decreased glomerular filtration rate, volume contraction, hypokalemia, and aldosterone excess. Clinical states associated with metabolic alkalosis are vomiting, mineralocorticoid excess, the adrenogenital syndrome, licorice ingestion, diuretic administration, and Bartter's and Gitelman's syndromes.

The two types of metabolic alkalosis (i.e., chloride-responsive, chloride-resistant) are classified based upon the amount of chloride in the urine. Chloride-responsive metabolic alkalosis involves urine chloride levels less than 10 mEq/L, and it is characterized by decreased extracellular fluid (ECF) volume and low serum chloride such as occurs with vomiting. This type responds to administration of chloride salt. Chloride-resistant metabolic alkalosis involves urine chloride levels more than 20 mEq/L, and it is characterized by increased ECF volume. As the name implies, this type resists administration of chloride salt. Ingestion of excessive oral alkali (usually milk plus calcium carbonate) and alkalosis complicating primary hyperaldosteronism are examples of chloride resistant alkalosis.

Many subjects with edematous states are treated with diuretics. Unfortunately, with continued therapy, the subject's bicarbonate level increases and progressive alkalosis may ensue. Diuretics cause metabolic alkalosis by several mechanisms, including (1) acute contraction of the extracellular fluid (ECF) volume (NaCl excretion without HCO₃—), thereby increasing the concentration of HCO₃⁻ in the ECF; (2) diuretic-induced potassium and chloride depletion; and (3) secondary aldosteronism. Continued use of the diuretic or either of the latter two factors will maintain the alkalosis.

The addition of an AA₁RA allows continued diuresis and maintained renal function without worsening the alkalosis. The AA₁RA inhibits the active resorption of HCO₃ across the proximal tubule of the kidney.

Thus, embodiments disclosed herein relate to a method of treating metabolic alkalosis, comprising identifying a subject in need thereof with BNP levels above about 450 pg/ml, e.g., above about 460 pg/mL, above about 470 pg/mL, above about 480 pg/mL, above about 500 pg/mL or more, or any number in between and/or subjects with NT-proBNP levels above about 1500 pg/ml, e.g., above about 1600 pg/mL, above about 1700 pg/mL, above about 1800 pg/mL, above about 1900 pg/mL, above about 2000 pg/mL or more, or any number in between, and administering a an adenosine A₁ receptor antagonist (AA₁RA) to said subject. The methods above can reduce the potential of related adverse events occurring, such as seizures or convulsions In certain embodiments, the subject is suffering from high altitude mountain sickness. In some embodiments, the subject has edema. In some of these embodiments, the subject may be on diuretic therapy. The diuretic may be a loop diuretic, proximal diuretic, or distal diuretic. In other embodiments, the subject suffers from acid loss through the subject's upper gastrointestinal tract, for example, through excessive vomiting. In still other embodiments the subject has ingested excessive oral alkali. The methods of the present invention can be practiced with any compound that antagonizes adenosine A₁ receptors.

Still other embodiments relate to the treatment of diabetic neuropathy in subjects with BNP levels above about 450 pg/ml, e.g., above about 460 pg/mL, above about 470 pg/mL, above about 480 pg/mL, above about 500 pg/mL or more, or any number in between and/or subjects with NT-proBNP levels above about 1500 pg/ml, e.g., above about 1600 pg/mL, above about 1700 pg/mL, above about 1800 pg/mL, above about 1900 pg/mL, above about 2000 pg/mL or more, or any number in between, by administering an AA₁RA to the subject. The methods disclosed herein can reduce the potential of related adverse events occurring, such as seizures or convulsions. Uncontrolled diabetes causes damage to many tissues of the body. Kidney damage caused by diabetes most often involves thickening and hardening (sclerosis) of the internal kidney structures, particularly the glomerulus (kidney membrane). Kimmelstiel-Wilson disease is the unique microscopic characteristic of diabetic nephropathy in which sclerosis of the glomeruli is accompanied by nodular deposits of hyaline.

The glomeruli are the site where blood is filtered and urine is formed. They act as a selective membrane, allowing some substances to be excreted in the urine and other substances to remain in the body. As diabetic nephropathy progresses, increasing numbers of glomeruli are destroyed, resulting in impaired kidney functioning. Filtration slows and protein, namely albumin, which is normally retained in the body, may leak in the urine. Albumin may appear in the urine for 5 to 10 years before other symptoms develop. Hypertension often accompanies diabetic nephropathy.

Diabetic nephropathy may eventually lead to the nephrotic syndrome (a group of symptoms characterized by excessive loss of protein in the urine) and chronic renal failure. The disorder continues to progress, with end-stage renal disease developing, usually within 2 to 6 years after the appearance of renal insufficiency with proteinuria.

The mechanism that causes diabetic nephropathy is unknown. It may be caused by inappropriate incorporation of glucose molecules into the structures of the basement membrane and the tissues of the glomerulus. Hyperfiltration (increased urine production) associated with high blood sugar levels may be an additional mechanism of disease development.

Diabetic nephropathy is the most common cause of chronic renal failure and end stage renal disease in the United States. About 40% of people with insulin-dependent diabetes will eventually develop end-stage renal disease. 80% of people with diabetic nephropathy as a result of insulin-dependent diabetes mellitus (IDDM) have had this diabetes for 18 or more years. At least 20% of people with non-insulin-dependent diabetes mellitus (NIDDM) will develop diabetic nephropathy, but the time course of development of the disorder is much more variable than in IDDM. The risk is related to the control of the blood-glucose levels. Risk is higher if glucose is poorly controlled than if the glucose level is well controlled.

Diabetic nephropathy is generally accompanied by other diabetic complications including hypertension, retinopathy, and vascular (blood vessel) changes, although these may not be obvious during the early stages of nephropathy. Nephropathy may be present for many years before nephrotic syndrome or chronic renal failure develops. Nephropathy is often diagnosed when routine urinalysis shows protein in the urine.

Current treatments for diabetic nephropathy include administration of angiotensin converting enzyme inhibitors (ACE Inhibitors) during the more advanced stages of the disease. Currently there is no treatment in the earlier stages of the disease since ACE inhibitors may not be effective when the disease is symptom-free (i.e., when the subject only shows proteinuria).

Although the mechanism implicated in early renal disease in diabetics is that of hyperglycemia, a potential mechanism may be related to the active reabsorption of glucose in the proximal tubule. This reabsorption is dependent in part on adenosine A₁ receptors.

AA₁RAs act on the afferent arteriole of the kidney to produce vasodilation and thereby improve renal blood flow in subjects with diabetes. This ultimately allows for increased GFR and improved renal function. In addition, AA₁RAs inhibit the reabsorption of glucose in the proximal tubule in subjects with newly diagnosed diabetic mellitus or in subjects at risk for the condition (metabolic syndrome).

Thus, provided herein are embodiments that relate to a method of treating diabetic nephropathy, comprising identifying a subject in need thereof with BNP levels above about 450 pg/ml, e.g., above about 460 pg/mL, above about 470 pg/mL, above about 480 pg/mL, above about 500 pg/mL or more, or any number in between and/or subjects with NT-proBNP levels above about 1500 pg/ml, e.g., above about 1600 pg/mL, above about 1700 pg/mL, above about 1800 pg/mL, above about 1900 pg/mL, above about 2000 pg/mL or more, or any number in between, and administering a an adenosine A₁ receptor antagonist (AA₁RA) to said subject. In certain embodiments the subject is pre-diabetic, whereas in other embodiments the subject is in early stage diabetes. In some embodiments the subject suffers from insulin-dependent diabetes mellitus (IDDM), whereas in other embodiments the subject suffers from non-insulin-dependent diabetes mellitus (NIDDM).

In certain embodiments, the methods of the present invention are used to prevent or reverse renal hypertrophy. In other embodiments, the methods of the present invention are used to prevent or reverse renal hyperplasia. In still other embodiments, the

Before people develop type II diabetes, i.e., NIDDM, they almost always have “pre-diabetes.” Pre-diabetic subjects have blood glucose levels that are higher than normal but not yet high enough to be diagnosed as diabetes. For instance, the blood glucose level of pre-diabetic subjects is between 110-126 mg/dL, using the fasting plasma glucose test (FPG), or between 140-200 mg/dL using the oral glucose tolerance test (OCTT). Blood glucose levels below 110 or 140, using FPG or OGTT, respectively, is considered normal, whereas individuals with blood glucose levels higher than 126 or 200, using FPG or OGTT, respectively, are considered diabetic. The methods of the present invention can be practiced with any compound that antagonizes adenosine A₁ receptors.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may, for example, include oral, rectal, transmucosal, or intestinal administration; parenteral delivery, including intramuscular, subcutaneous, intravenous, intra-arterially, intramedullary injections, as well as intrathecal, direct intraventricular, intraperitoneal, intranasal, or intraocular injections.

Alternately, one may administer the compound in a local rather than systemic manner, for example, via injection of the compound directly in the renal or cardiac area, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions of the present invention may be manufactured in a manner that is itself known, e.g., by means of conventional mixing, dissolving, granulating, dragee-making, levigating, emulsifying, encapsulating, entrapping or tabeleting processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Some emulsions used in solubilizing and delivering the xanthine derivatives described above are discussed in U.S. Pat. No. 6,210,687, which is incorporated by reference herein in its entirety, including any drawings.

Many of the compounds used in the pharmaceutical combinations of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions of the present invention can be chosen by the individual physician in view of the patient's condition. (See e.g., Fingi et al. 1975, in “The Pharmacological Basis of Therapeutics”, Ch. 1 p. 1). Typically, the dose range of the composition administered to the patient can be from about 0.01 to 1000 mg/kg of the patient's body weight. The dosage may be a single one or a series of two or more given in the course of one or more days, as is needed by the patient.

The daily dosage regimen of KW-3902 for an adult human patient may be, for example, an oral dose of between 0.1 mg and 500 mg, preferably between 1 mg and 250 mg, e.g. 5 to 200 mg or an intravenous, subcutaneous, or intramuscular dose of between 0.01 mg and 500 mg, preferably between 0.1 mg and 200 mg, e.g. 1 to 100 mg of the pharmaceutical compositions of the present invention or a pharmaceutically acceptable salt thereof calculated as the free base, the composition being administered 1 to 4 times per day. Alternatively the compositions of the invention may be administered by continuous intravenous infusion, preferably at a dose of up to 400 mg per day. Thus, the total daily dosage by oral administration will be in the range 1 to 2000 mg and the total daily dosage by parenteral administration will be in the range 0.1 to 400 mg. Suitably the compounds will be administered for a period of continuous therapy, for example for a week or more, or for months or years.

In certain embodiments, KW-3902 is administered in conjunction with a diuretic. In these aspects, the dose of the diuretic is that which constitutes standard diuretic therapy. Those of skill in the art know what dosage of diuretics to administer to a patient in need thereof. However, because of the diuretic effect of KW-3902, the need for higher doses of the diuretic are eliminated when KW-3902 is administered to a patient in conjunction with the diuretic.

Dosage amount and interval may be adjusted individually to provide plasma levels of the active moiety that are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

Having now generally described the invention, the same will become better understood by reference to certain specific examples which are included herein for purposes of illustration only and are not intended to be limiting unless other wise specified. All referenced publications and patents are incorporated, in their entirety by reference herein.

### EXAMPLES

### Example 1

### KW-3902 Improves Serum Creatinine Levels, Dyspnea and Delays Worsening Heart Failure in Subjects with Acute CHF and Renal Impairment

More than 300 subjects hospitalized due to acute CHF requiring intravenous diuretic therapy to treat fluid overload, and presenting with creatinine clearance values between 20 to 80 mL/min were identified. The subjects were randomized to receive either placebo, or 10 mg, 20 mg or 30 mg intravenous KW-3902 per day. The subjects' BNP levels, NT-Pro-BNP, serum creatinine, weight, and NYHA Class were measured.

On day 1, KW-3902 (or placebo) was co-administered with intravenous furosemide (LASIX™). The specified dose of KW-3902 (or placebo) was infused over a four hour time period. Subjects received therapy for up to three days. Serum creatinine, weight, dyspnea, and worsening heart failure were assessed daily by the investigator on days 1 through 14. Worsening heart failure was investigator determined and required an increase in dose or reinstitution of LASIX, mechanical respiratory or circulatory assist measures, administration of intravenous positive vasopressors, or administration of intravenous vasodilators.

Subjects were categorized as “Failure,” if after Day 7, the subject died, was readmitted to the hospital for heart failure, exhibited worsening heart failure requiring rescue therapy, or exhibited an increase in serum creatinine levels greater than or equal to 0.3 mg/dL at the earlier of hospital discharge or Day 7, compared to the subject's baseline creatinine levels (Day 1).

Subjects were categorized as “Success” if the subject was not characterized as Failure, and if on either day 2 or 3, the subject reported markedly or moderately improved dyspnea, and the investigator reported that the subject had improved such that intravenous diuretic therapy could be converted to oral diuretic therapy.

Subjects ere categorized as “Unchanged” if the subject was not categorized as a “Success” or “Failure.”

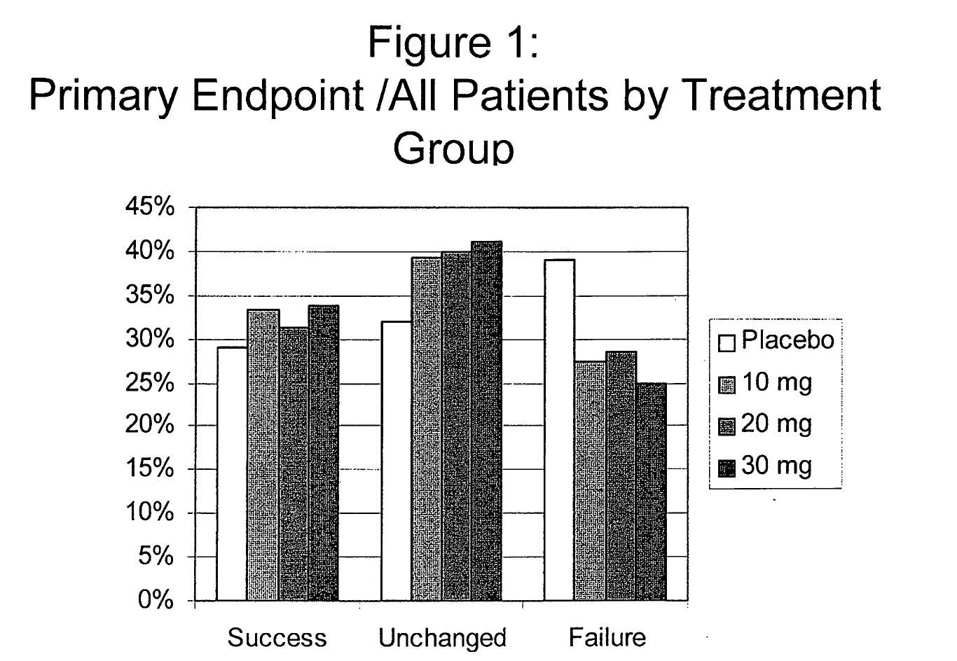

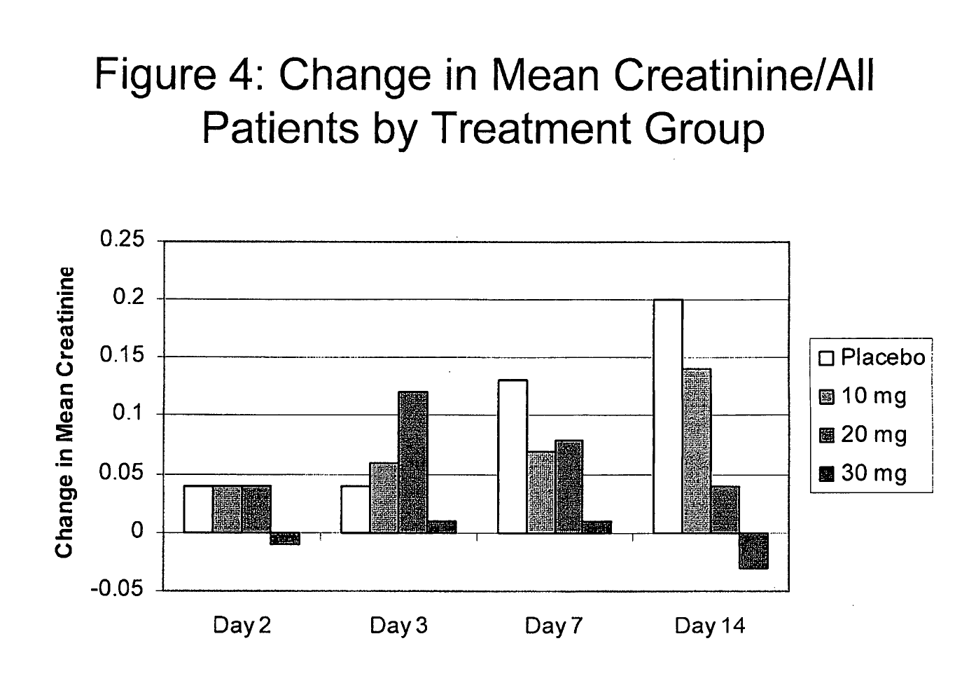

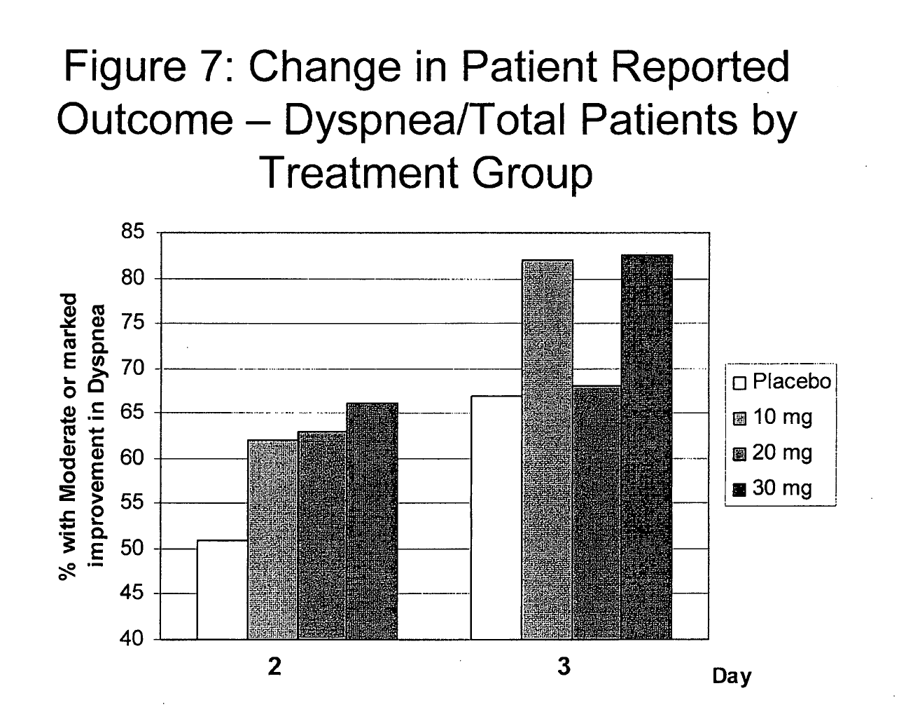

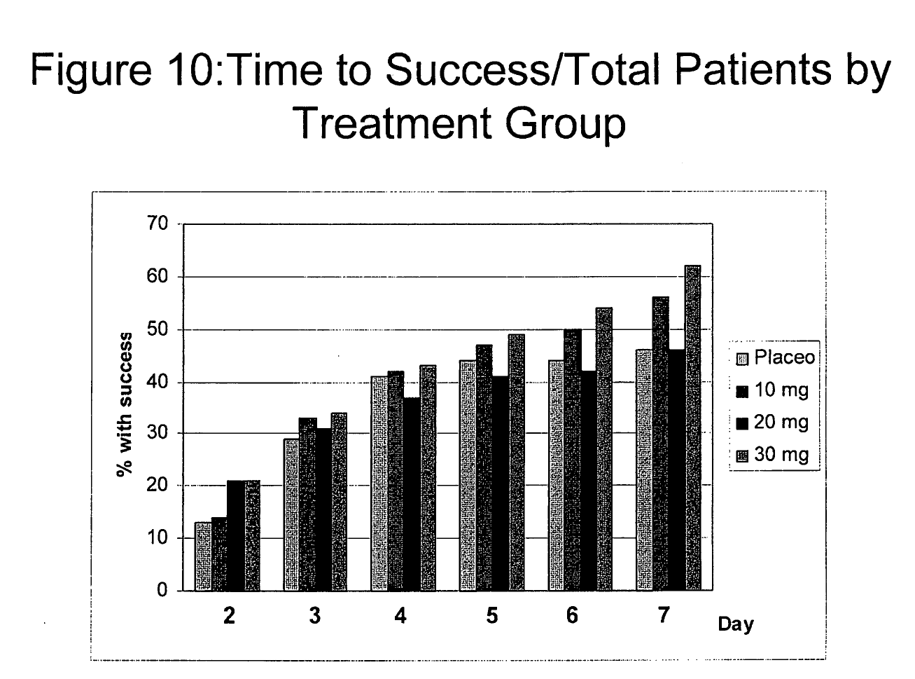

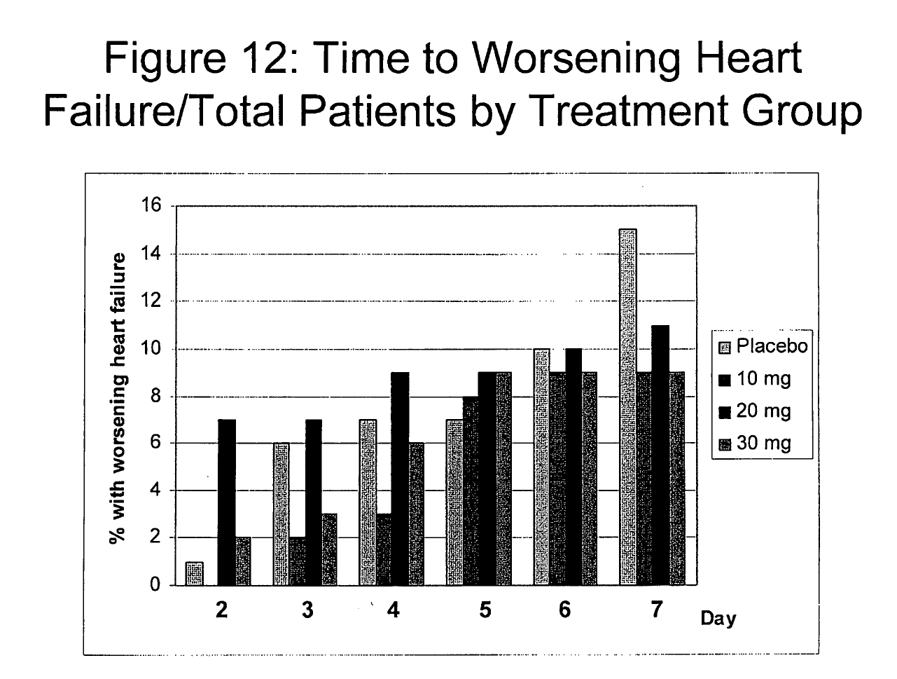

The data from the study are presented in FIGS. 1, 4, 7, 10, and 12. The percentage of subjects classified as “Failure” was reduced in all three KW-3902 treatment groups as compared to subjects receiving placebo (FIG. 1). Subjects receiving placebo exhibited a greater increase in serum creatinine levels over time. Notably, subjects that received 30 mg KW-3902 showed an overall decrease in serum creatinine levels, indicative of an improvement in renal function at Day 14, whereas subjects that received placebo showed a mean increase in serum creatinine levels at Day 14, indicative of worsening renal function (FIG. 4). Notably, though KW-3902 was only administered over Days 1, 2, and 3, the improvement in serum creatinine levels was observed on Day 14, demonstrating that KW-3902 has a persistent effect on renal function. A greater percentage of subjects reported moderate or marked improvement in dyspnea in groups that received KW-3902 therapy versus in groups that received placebo (FIG. 7). FIG. 10 shows the percentage of subjects characterized as “Success” as described above over time. By Day 7, a higher percentage of the group of subjects treated with 30 mg KW-3902 that were ultimately characterized as “Success” were so characterized at Day 7, compared to the percentage of “Success” subjects that were treated with placebo. In other words, subjects treated with 30 mg KW-3902 improved more quickly than subjects treated with placebo. FIG. 12 shows the percentage of subjects identified as having worsening heart failure in each treatment group over time. By Day 7 a higher percentage of subjects were identified as having worsening heart failure in the placebo treatment group compared to the groups that were treated with KW-3902.

### Example 2

### Treatment of Individuals with BNP Levels Greater than 500 pg/mL

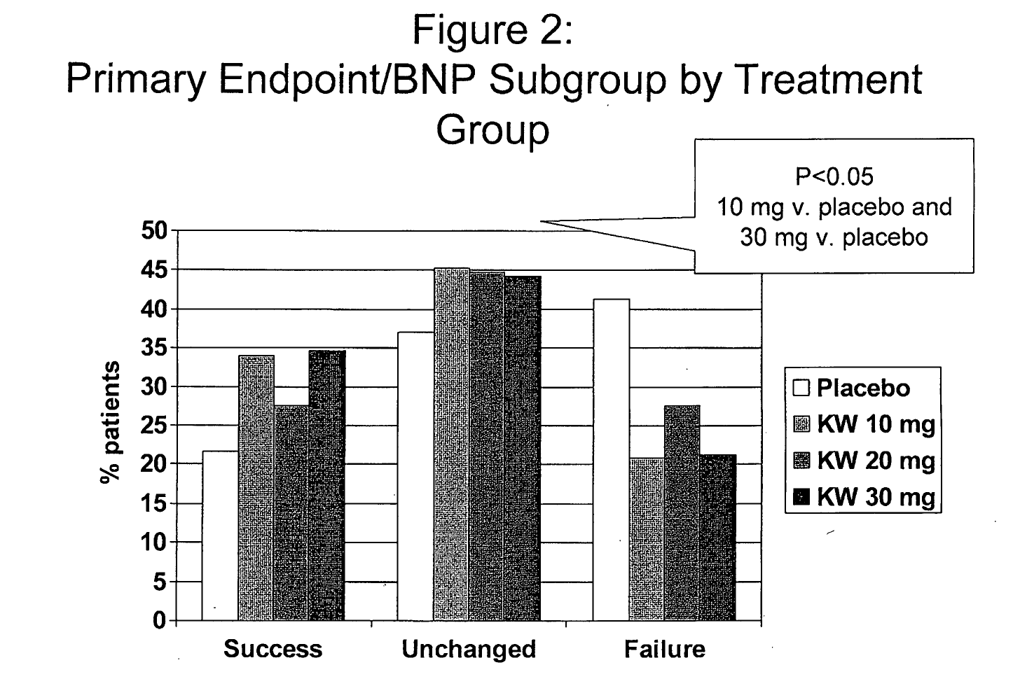

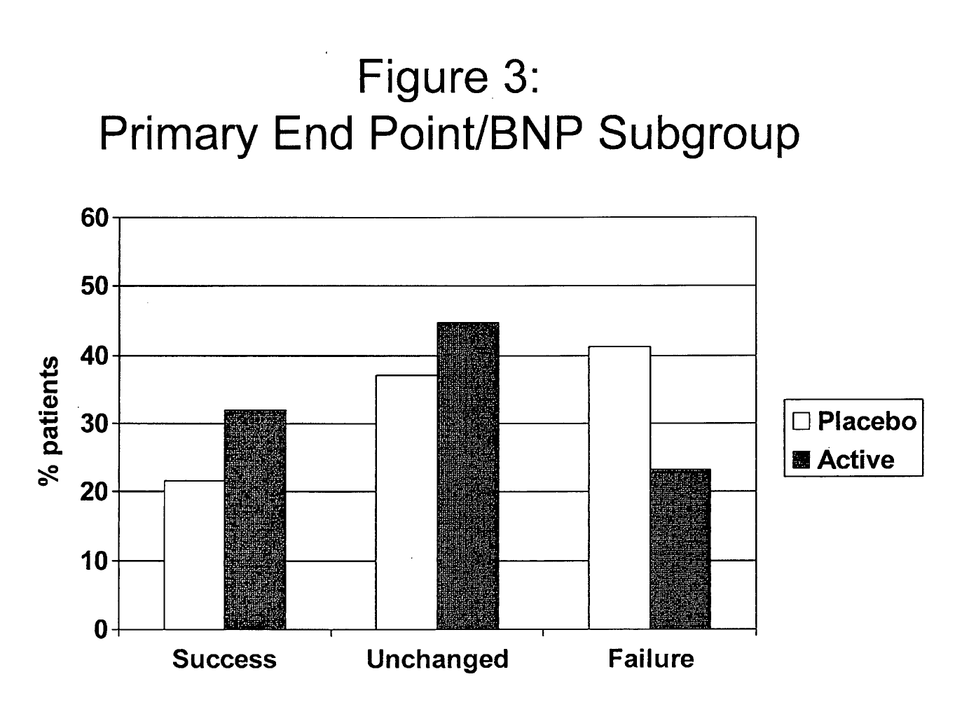

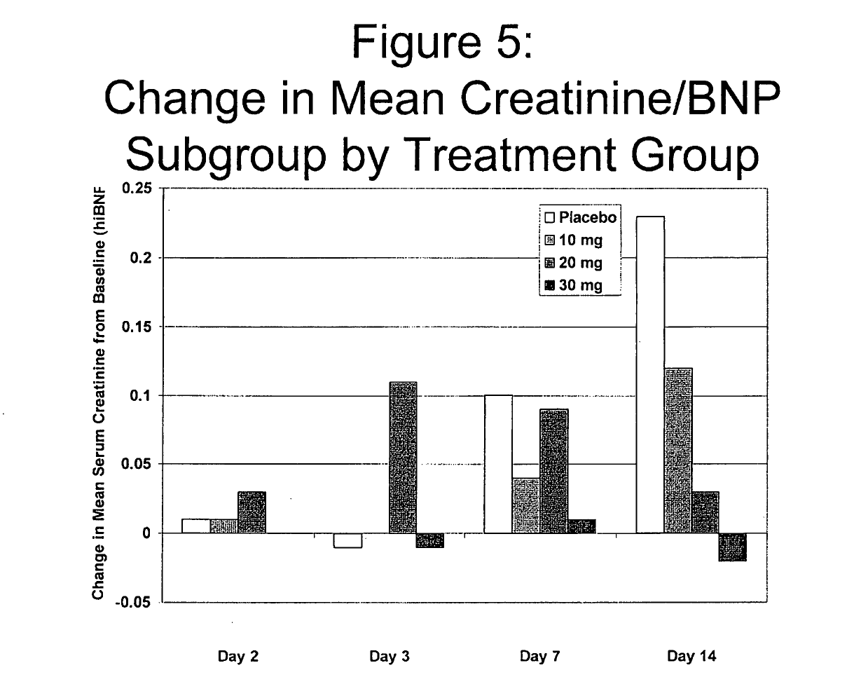

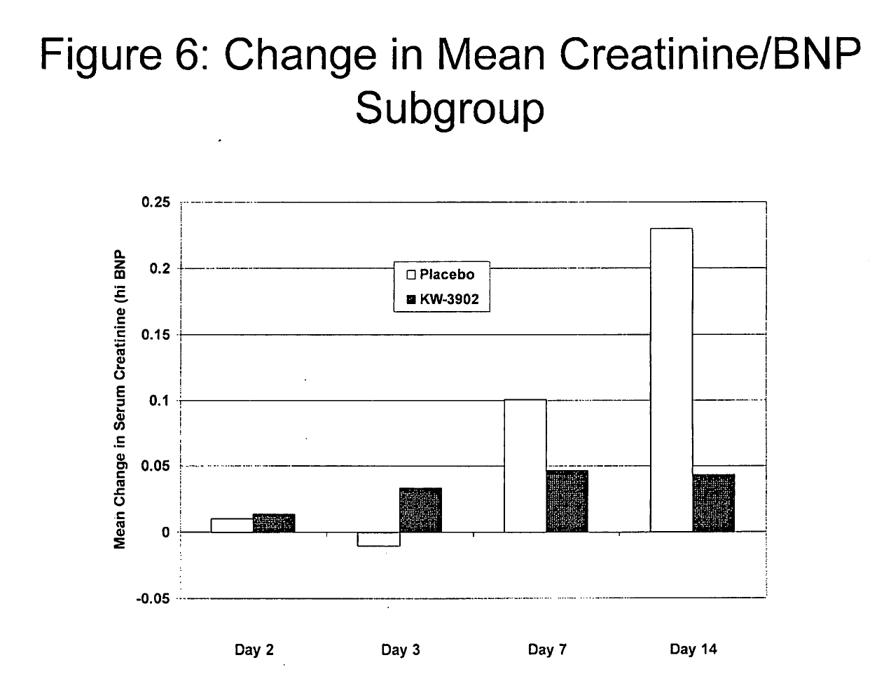

To compare the therapeutic effect of KW-3902 on individuals with different levels of BNP and or NT-Pro-BNP, the results of the trial discussed in Example 1 were separately analyzed to distinguish subjects with BNP levels exceeding 500 pg/mL. Approximately 75% of the subjects described in Example 1 fell within the subgroup having BNP levels above 500 pg/mL. (“BNP subgroup”) The difference in the percentage of subjects classified as “Success” and those classified as “Failure” is more dramatic between the treatment groups and the placebo in the BNP subgroup. (FIG. 1, FIG. 2, FIG. 3) Similarly, the change in mean serum creatinine levels between subjects receiving placebo versus KW-3902 is more dramatic in the BNP subgroup. (FIG. 4, FIG. 5, FIG. 6).

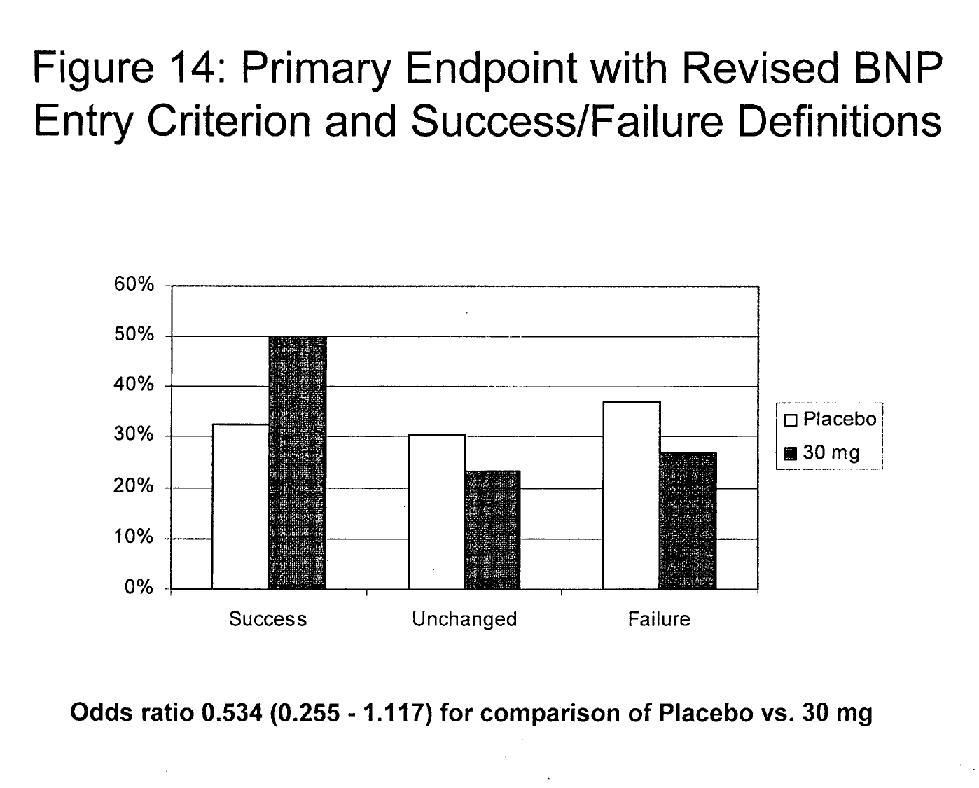

FIG. 14 shows that when the characterization of “Success” is modified in the BNP subgroup, such a subject is characterized as “Success” if the subject was not characterized as Failure, and if on either day 2 or 3, the subject reported markedly or moderately improved dyspnea, the difference between individuals treated with 30 mg KW-902 compared to placebo is much more dramatic. Further, greater than 65% of individuals treated with KW-3902 are characterized as “Success” following treatment with 30 mg/KW-3902.

### Example 3

### Treatment of Individuals with Fluid Overload and Renal Impairment

A subject with fluid overload, as manifested by peripheral edema, dyspnea, and/or other signs or symptoms presents to the hospital, clinic, or doctor's office. The subject also shows some degree of renal impairment. The subject's BNP levels are greater than 500 pg/mL, and/or the subject's NT-Pro-BNP levels are above 2000 pg/mL. In addition to standard of care therapy which would include IV diuretics, e.g., IV furosemide, bumetanide and/or oral metolazone, the subject is also given an amount of KW-3902 between 2.5 mg and 60 mg in injectable form. The subject is administered the dose of KW-3902 and 40 mg of furosemide at 24 hour intervals or more frequently as needed. The subject's fluid intake and output, urine volume, serum and urine creatinine levels, electrolytes and cardiac function are monitored.

At the discretion of the attending physician, the dosage of KW-3902 can be increased or decreased during the treatment. In addition, the dosage of furosemide can be increased to 60 mg, 80 mg, 100 mg, 120 mg, 140 mg, or 160 mg either during the treatment or as the initial dose, or furosemide can be given as a continuous infusion.

### Example 4

### Treatment of Individuals Refractory to Standard IV Diuretic Therapy

Subjects presenting with congestive heart failure who were refractory to high dose diuretic therapy, who exhibit BNP levels above 500 pg/mL and/or NT-Pro-BNP levels exceeding 2000 pg/mL, and who have an estimated creatinine clearance between 20 mL/min and 80 mL/min are identified. Identified subjects are administered between 2.5 and 100 mg KW-3902, and preferably about 30 mg KW-3902 IV. Changes in urine output and creatinine clearance rate are measured.

A hospitalized subject who has been treated with maximum amounts of IV diuretic and is still symptomatic, fluid overloaded, or whose urine output is less than fluid intake is evaluated for further treatment. A dose of KW-3902 between about 2.5 mg and 100 mg, preferably about 30 mg, in injectable form is infused through the IV line. The subject receives continued treatment with furosemide, and also receives doses of KW-3902 at 6 hour intervals, or more or less frequently as needed. The subject's fluid intake and output, urine volume, serum and urine creatinine levels, electrolytes and cardiac function are monitored.

At the discretion of the attending physician, the dosage of KW-3902 can be increased or decreased during the treatment as needed. In addition, the dosage of furosemide can be increased to 60 mg, 80 mg, 100 mg, 120 mg, 140 mg, or 160 mg either during the treatment or as the initial dose, or furosemide can be given as a continuous infusion.

### Example 5

### Treatment of Individuals Refractory to Standard IV Diuretic Therapy

A hospitalized subject who has been treated with maximum amounts of IV diuretic and is still symptomatic, fluid overloaded, or whose urine output is less than fluid intake is evaluated for further treatment. The subject's BNP levels are greater than 500 pg/mL, and/or the subject's NT-Pro-BNP levels are above 2000 pg/mL. A dose of about 2.5 mg to about 100 mg KW-3902, preferably about 30 mg KW-3902 in injectable form is infused through the IV line. The subject receives continued treatment with furosemide, and also receives doses of KW-3902 at 6 hour intervals, or more or less frequently as needed. The subject's fluid intake and output, urine volume, serum and urine creatinine levels, electrolytes and cardiac function are monitored.

At the discretion of the attending physician, the dosage of KW-3902 can be increased or decreased during the treatment as needed. In addition, the dosage of furosemide can be increased to 60 mg, 80 mg, 100 mg, 120 mg, 140 mg, or 160 mg either during the treatment or as the initial dose, or furosemide can be given as a continuous infusion.

### Example 6

### Treatment of Individuals with Fluid Overload

A subject with fluid overload, as manifested by peripheral edema, dyspnea, and/or other signs or symptoms presents to the hospital, clinic, or doctor's office. The subject's BNP levels are greater than 500 pg/mL, and/or the subject's NT-Pro-BNP levels are above 2000 pg/mL. In addition to standard of care therapy which would include IV diuretics, e.g., IV furosemide, bumetanide and/or oral metolazone, the subject is also given a dose of about 2.5 mg to about 100 mg, preferably about 30 mg of KW-3902 in injectable form. The subject is administered doses of KW-3902 and 40 mg of furosemide at 24 hour intervals, or furosemide can be given as a continuous infusion. The subject's fluid intake and output, urine volume, serum and urine creatinine levels, electrolytes and cardiac function are monitored.

At the discretion of the attending physician, the dosage of KW-3902 can be increased or decreased during the treatment as needed. In addition, the dosage of furosemide can be increased to 60 mg, 80 mg, 100 mg, 120 mg, 140 mg, or 160 mg either during the treatment or as the initial dose, or furosemide can be given as a continuous infusion.

### Example 7

### Treatment of Individuals with Fluid Overload and Impaired Renal Function

A subject with fluid overload, as manifested by peripheral edema, dyspnea, and/or other signs or symptoms presents himself to the physician's office or clinic. The subject's BNP levels are greater than 500 pg/mL, and/or the subject's NT-Pro-BNP levels are above 2000 pg/mL. The subject has been on a therapy regimen that includes oral diuretics and, in addition, to needing a higher dose of diuretics to manage his/her fluid balance, the subject is now showing impaired renal function. The subject is prescribed 5 mg of KW-3902 to be taken orally, once daily, concurrent with other diuretic therapy. The subject's fluid intake and output, urine volume, serum and urine creatinine levels, electrolytes and cardiac function are monitored.

At the discretion of the attending physician, the dosage of KW-3902 can be increased or decreased during the treatment as needed. In addition, the dosage of furosemide can be increased to 60 mg, 80 mg, 100 mg, 120 mg, 140 mg, or 160 mg either during the treatment or as the initial dose, or furosemide can be given as a continuous infusion.

### Example 8

### Treatment of Individuals with Fluid Overload

A subject with fluid overload, as manifested by peripheral edema, dyspnea, and/or other signs or symptoms presents to the physician's office or clinic. The subject's BNP levels are greater than 500 pg/mL, and/or the subject's NT-Pro-BNP levels are above 2000 pg/mL. The subject has been on a therapy regimen that includes oral diuretics and needs a higher dose of diuretics to manage his/her fluid balance. To delay or prevent the onset of renal impairment and/or to delay the need to use higher dosages of standard diuretics, the subject is prescribed about 2.5 to about 100 mg of KW-3902, preferably about 30 mg KW-3902 to be taken orally, once daily, concurrent with their diuretic therapy. The subject's fluid intake and output, urine volume, serum and urine creatinine levels, electrolytes and cardiac function are monitored.

At the discretion of the attending physician, the dosage of KW-3902 can be increased or decreased during the treatment as needed. In addition, the dosage of furosemide can be increased to 60 mg, 80 mg, 100 mg, 120 mg, 140 mg, or 160 mg either during the treatment or as the initial dose, or furosemide can be given as a continuous infusion.

### Example 9

### Treatment of Individuals with Congestive Heart Failure

A subject with congestive heart failure presents to the physician's office or clinic. The subject's BNP levels are greater than 500 pg/mL, and/or the subject's NT-Pro-BNP levels are above 2000 pg/mL. The subject is put on a therapy regimen that includes oral diuretics to manage his/her fluid balance. To delay or prevent the onset of renal impairment and/or to delay the need to use higher dosages of standard diuretics, the subject is also prescribed 5 mg of KW-3902 to be taken orally, once daily, concurrent with their diuretic therapy. The subject's fluid levels, urine volume, serum and urine creatinine levels, electrolytes and cardiac function are monitored.

At the discretion of the attending physician, the dosage of KW-3902 can be increased or decreased during the treatment as needed. In addition, the dosage of furosemide can be increased to 60 mg, 80 mg, 100 mg, 120 mg, 140 mg, or 160 mg either during the treatment or as the initial dose, or furosemide can be given as a continuous infusion.

### Example 10

### Improving Health Outcomes for of Individuals with Congestive Heart Failure

A subject with congestive heart failure presents to the physician's office or clinic. The subject's BNP levels are greater than 500 pg/mL, and/or the subject's NT-Pro-BNP levels are above 2000 pg/mL. The subject is put on a therapy regimen that includes oral diuretics to manage his/her fluid balance. To improve overall health outcomes (i.e., morbidity or mortality rates due to CHF), the subject is also prescribed 5 mg of KW-3902 to be taken orally, once daily, concurrent with their diuretic therapy, or similar doses of KW-3902 is administered to the subject intravenously. The subject's fluid levels, urine volume, serum and urine creatinine levels, electrolytes and cardiac function are monitored.

At the discretion of the attending physician, the dosage of KW-3902 can be increased or decreased during the treatment as needed. In addition, the dosage of furosemide can be increased to 60 mg, 80 mg, 100 mg, 120 mg, 140 mg, or 160 mg either during the treatment or as the initial dose, or furosemide can be given as a continuous infusion.

What is claimed is:

1. A method, comprising:
identifying an individual in need of therapy to improve, maintain, or restore renal function;
ascertaining that the individual has brain natriuretic peptide levels of at least 500 pg/mL or N-terminal pro-brain natriuretic peptide levels of at least 2000 pg/mL; and
administering to the individual a therapeutically effective amount of KW-3902 or a pharmaceutically acceptable salt, amide, prodrug ester or metabolite thereof to improve, maintain, or restore renal function.

2. The method of claim 1 wherein the individual has congestive heart failure (CHF).

3. The method of claim 2, wherein said CHF is acute CHF.

4. The method of claim 2, wherein the individual is refractory to standard diuretic therapy.

5. The method of claim 1, further comprising administering to the individual a non-adenosine modifying diuretic.

6. The method of claim 4, further comprising administering to the individual a non-adenosine modifying diuretic.

7. The method of claim 1, wherein said therapeutically effective amount of KW-3902 or pharmaceutically acceptable salt, amide, prodrug ester, or metabolite thereof is sufficient to maintain the individual's creatinine clearance rate.

8. The method of claim 1, wherein said therapeutically effective amount of KW-3902 or pharmaceutically acceptable salt, amide, prodrug ester, or metabolite thereof is sufficient to increase the individual's creatinine clearance rate.

9. The method of claim 1, further comprising measuring the creatinine clearance in the individual.

10. The method of claim 1, wherein said therapeutically effective amount of KW-3902 is between about 2.5 mg and about 70 mg per day.

11. The method of claim 10, wherein said therapeutically effective amount of KW-3902 is about 30 mg per day.

12. The method of claim 5, wherein said non-adenosine modifying diuretic is selected from the group consisting of a loop diuretic, a proximal diuretic and a distal diuretic.

13. The method of claim 6, wherein said non-adenosine modifying diuretic is selected from the group consisting of a loop diuretic, a proximal diuretic and a distal diuretic.

14. The method of claim 12, wherein said non-adenosine modifying diuretic is furosemide.

15. The method of claim 13, wherein said non-adenosine modifying diuretic is furosemide.

16. An method, comprising:
identifying an individual with heart failure and mild to severe renal impairment;
ascertaining that the individual has brain natriuretic peptide levels of at least 500 pg/mL or N-terminal pro-brain natriuretic peptide levels of at least 2000 pg/mL; and
administering to the individual a therapeutically effective amount of KW-3902 or a pharmaceutically acceptable salt, amide, prodrug ester or metabolite thereof to treat heart failure and mild to severe renal impairment.

17. The method of claim 16, wherein the individual has congestive heart failure (CHF).

18. The method of claim 17, wherein said CHF is acute CHF.

19. The method of claim 16, wherein the individual has dyspnea.

20. The method of claim 17, wherein the individual is refractory to standard diuretic therapy.

21. The method of claim 16, further comprising administering to the individual a non-adenosine modifying diuretic.

22. The method of claim 20, further comprising administering to the individual a non-adenosine modifying diuretic.

23. The method of claim 16, wherein said therapeutically effective amount of KW-3902 or pharmaceutically acceptable salt, ester, amide, prodrug, or metabolite thereof is sufficient to maintain the individual's creatinine clearance rate.

24. The method of claim 16, wherein said therapeutically effective amount of KW-3902 or pharmaceutically acceptable salt, ester, amide, prodrug, or metabolite thereof is sufficient to increase the individual's creatinine clearance rate.

25. The method of claim 16, further comprising measuring the creatinine clearance rate in the individual.

26. The method of claim 16, wherein said therapeutically effective amount of KW-3902 is between about 2.5 mg and about 70 mg per day.

27. The method of claim 26, wherein said therapeutically effective amount of KW-3902 is about 30 mg per day.

28. The method of claim 21, wherein said non-adenosine modifying diuretic is selected from the group consisting of a loop diuretic, a proximal diuretic and a distal diuretic.

29. The method of claim 22, wherein said non-adenosine modifying diuretic is selected from the group consisting of a loop diuretic, a proximal diuretic and a distal diuretic.

30. The method of claim 21, wherein said non-adenosine modifying diuretic is furosemide.

31. The method of claim 22, wherein said non-adenosine modifying diuretic is furosemide.

32. A method for treating a patient for acute fluid overload, comprising:
identifying a patient in need of short-term hospitalization to treat acute fluid overload with brain natriuretic peptide levels of at least 500 pg/mL or N-terminal pro-brain natriuretic peptide levels of at least 2000 pg/mL;
hospitalizing the patient;
administering diuretic therapy to the patient while hospitalized, wherein said diuretic therapy comprises a non adenosine-modifying diuretic; and
administering to the patient an amount of KW-3902 or a pharmaceutically acceptable salt, prodrug ester, amide, or metabolite thereof, effective to accelerate removal of excess fluid from the patient in comparison to said diuretic therapy alone.

33. The method of claim 32, wherein said therapeutically effective amount of KW-3902 is between about 10 mg and about 40 mg per day.

34. The method of claim 33, wherein said therapeutically effective amount of KW-3902 is about 30 mg per day.

35. The method of claim 32, wherein said non-adenosine modifying diuretic is selected from the group consisting of a loop diuretic, a proximal diuretic and a distal diuretic.

36. The method of claim 35, wherein said non-adenosine modifying diuretic is furosemide.

37. A method of maintaining or improving renal function in an individual with stable congestive heart failure (CHF) who is also receiving chronic diuretic therapy, comprising
ascertaining that the individual has brain natriuretic peptide levels of at least 500 pg/mL or N-terminal pro-brain natriuretic peptide levels of at least 2000 pg/mL; and
administering to the individual a therapeutically effective amount of an adenosine A1 receptor antagonist (AA₁RA) at intervals of about four day to about monthly, wherein the individual simultaneously continues said chronic diuretic therapy throughout the course of treatment with said AA₁RA.

38. The method of claim 37, wherein the AA₁RA is KW-3902 and the therapeutically effective amount of KW-3902 is between about 2.5 to about 70 mg/dose.

39. The method of claim 38, wherein the therapeutically effective amount of KW-3902 is about 30 mg/dose.

40. The method of claim 37, wherein the AA₁RA is administered at intervals of about 4 to 14 days.

41. The method of claim 37, wherein the AA₁RA is administered at intervals of about 7 to 30 day.

42. A method of treating an individual experiencing mild renal impairment, wherein the individual is undergoing diuretic therapy, comprising
ascertaining that the individual has brain natriuretic peptide levels of at least 500 pg/mL or N-terminal pro-brain natriuretic peptide levels of at least 2000 pg/mL; and
administering to the individual a therapeutically effective amount of an AA₁RA on a bi-weekly to monthly basis.

43. The method of claim 43, wherein said AA₁RA is selected from the group consisting of KW-3902, BG-9719, BG-9928, or a pharmaceutically acceptable salt, amide, prodrug ester, or metabolite thereof.

44. The method of claim 43, wherein said AA₁RA is KW-3902 or a pharmaceutically acceptable salt, amide, prodrug ester, or metabolite thereof.

45. The method of claim 44, wherein said therapeutically effective amount of KW-3902 is between about 2.5 to about 70 mg/dose.

46. The method of claim 45, wherein said therapeutically effective amount of KW-3902 is about 30 mg/dose.

47. The method of claim 42, wherein said AA₁RA is administered at intervals of about 4 to 14 days.

48. The method of claim 42, wherein said AA₁RA is administered at intervals of about 7 to 30 days.

49. A method for slowing or reversing renal impairment in a individual, comprising
selecting an individual with an existing or developing renal impairment and having brain natriuretic peptide levels of at least 500 pg/mL or N-terminal pro-brain natriuretic peptide levels of at least 2000 pg/mL; and
administering to the individual an effective periodic dose of an AA₁RA between about once every four days and about once every month.

50. The method of claim 49, wherein said AA₁RA is selected from the group consisting of KW-3902, BG-9719, BG-9928, or a pharmaceutically acceptable salt, amide, prodrug ester, or metabolite thereof.

51. The method of claim 50, wherein said AA₁RA is KW-3902 or a pharmaceutically acceptable salt, amide, prodrug ester, or metabolite thereof.

52. The method of claim 51, wherein said therapeutically effective amount of KW-3902 is between about 2.5 to about 70 mg/dose.

53. The method of claim 52, wherein said therapeutically effective amount of KW-3902 is about 30 mg/dose.

54. The method of claim 49, wherein said AA₁RA is administered at intervals of about 4 to 14 days.

55. The method of claim 49, wherein said AA₁RA is administered at intervals of about 7 to 30 days.
